(19) United States
(12) Patent Application Publication
(10) Pub. No.: US 2022/0146534 A1
(43) Pub. Date: May 12, 2022

(54) Biomarkers for CNS Disease Modification

(71) Applicant: Yeda Research and Development, Co., Ltd., Rehovot (IL)

(72) Inventors: Michal Eisenbach-Schwartz, Rehovot (IL); Hila Ben-Yehuda, Rehovot (IL); Michal Arad, Rehovot (IL); Tommaso Croese, Rehovot (IL); Javier Maria Peralta Ramos, Rehovot (IL); Giulia Castellani, Rehovot (IL); Neta Rosenzweig, Rehovot (IL)

(73) Assignee: Yeda Research and Development, Co., Ltd., Rehovot (IL)

(21) Appl. No.: 17/423,769

(22) PCT Filed: Jan. 16, 2020
(86) PCT No.: PCT/IL2020/050072

§ 371 Date: Jul. 16, 2021

Related U.S. Application Data
(60) Provisional application No. 62/792,978, filed on Jan. 16, 2019.

(51) Int. Cl. G01N 33/68; G01N 33/50

(52) U.S. Cl. CPC G01N 33/6896; G01N 33/5047; G01N 2800/52; G01N 2333/70596; G01N 33/5091

(57) ABSTRACT

A method for predicting whether a patient diagnosed with a disease, disorder, condition or injury of the CNS is likely to be responsive or non-responsive to treatment with an immune checkpoint modulator is provided, wherein said method comprises determining ex vivo, in a blood sample obtained from the patient, or in a fraction thereof, a biomarker selected from: (a) the level of a monocyte subpopulation expressing CCR2, CD204 or a combination thereof, or CCR2 and a marker selected from igf1, lyve1, Stab-1, Siglec1 and Mrc1, or any combination thereof, (b) the ratio of the level of a monocyte subpopulation (CD14⁺ cells) expressing CCR2^{high}CX3CR1^{low} to a monocyte subpopulation expressing CCR2^{low}CX3CR1^{high}; (c) the level of a CCR2 agonist; and (d) the level of a CCR2 antagonist.

This application is a 35 U.S.C. § 371 U.S. national stage patent application which claims the benefit of priority and is entitled to the filing date of International Patent Application PCT/IL2020/050072, filed Jan. 16, 2020, an international patent application which claims the benefit of priority and is entitled to the filing date pursuant to 35 U.S.C. § 119(e) of U.S. Provisional Patent Application 62/792,978, filed Jan. 16, 2019, the content of each of which is hereby incorporated by reference in its entirety.

### FIELD OF THE INVENTION

The present invention relates in general to prognostic markers for central nervous system (CNS) disease, such as Alzheimer's disease.

### BACKGROUND OF THE INVENTION

Alzheimer's disease (AD) is a heterogeneous disorder with multiple etiologies. Harnessing the immune system by blocking the programmed cell death receptor (PD)-1 pathway in an amyloid beta mouse model is known to evoke a sequence of immune responses that lead to disease modification.

Over the last two decades, it has become clear that systemic immune cells are important players in brain maintenance and repair, with implications to brain aging and neurodegenerative conditions¹⁻¹². Moreover, systemic immune deficiency has been associated with cognitive dysfunction^{4,13}, behavioral dysfunction¹⁴ and reduced ability to cope with neurodegenerative conditions, including Amyotrophic lateral sclerosis (ALS)¹⁰ and AD^{8,9,12}. In line with this, boosting recruitment of monocyte-derived macrophages to sites of brain pathology in several mouse models of AD, such as the amyloid-beta driven AD mouse model, 5×FAD¹⁵ as well as the animal model of tau pathology, expressing the human-tau gene with two mutations associated with fronto-temporal dementia (DM-hTAU)¹⁶, results in reduced brain pathology, in general, and reduced plaque burden, in particular^{8,9,12} (WO 2015/136541; WO 2017/009829; WO 2018/047178).

There remains a need for surrogate markers and companion diagnostic for methods for treating CNS disease, such as Alzheimer's disease.

### SUMMARY OF INVENTION

In one aspect, the present invention provides a method for predicting whether a patient diagnosed with a disease, disorder, condition or injury of the CNS is likely to be responsive or non-responsive to treatment with an immune checkpoint modulator, said method comprising determining ex vivo, in a blood sample obtained from the patient, or in a fraction thereof, a biomarker selected from: (a) the level of a monocyte subpopulation (CD14⁺ cells) expressing C—C chemokine receptor type 2 (CCR2, a.k.a. CD192) or macrophage scavenger receptor 1 (MSR-1, a.k.a. SRA1, SCARA1 and CD204) or a combination thereof, or CCR2 and a marker selected from insulin-like growth factor-1 (igf1), lymphatic endothelium-specific hyaluronan receptor (lyve1), scavenger receptor stabilin-1 (Stab-1), sialic acid binding Ig like lectin 1 (Siglec1) and mannose receptor C-type (Mrc1), or any combination thereof; (b) the ratio of the level of a monocyte subpopulation (CD14⁺ cells) expressing CCR2^{high}CX3CR1^{low} to a monocyte subpopulation (CD14⁺ cells) expressing CCR2^{low}CX3CR1^{high}; (c) the level of a CCR2 agonist selected from CCL2, CCL7, CCL13, CCL8, CCL11 and CCL16; and (d) the level of a CCR2 antagonist selected from CCL24 and CCL26, wherein an equal or increased level of said biomarker (a) to (c) or a decreased level of said biomarker (d) in the blood sample, or a fraction thereof, as compared to a first or a second reference indicates that the patient is likely to be responsive to treatment with said immune checkpoint modulator, and an equal or decreased level of any one of said biomarker (a) to (c) or an increased level of said biomarker (d) in the blood sample, or a fraction thereof, as compared to said first or second reference indicates that the patient is likely to be non-responsive to treatment with said immune checkpoint modulator, and in case the blood sample is obtained from the patient prior to treatment with said immune checkpoint modulator, the level of said biomarker in said blood sample, or fraction thereof, is compared with the first reference, which is the level of said biomarker in blood, or a fraction thereof, of a responder patient population before start of treatment with said immune checkpoint modulator; or in case the blood sample is obtained from the patient after treatment with said immune checkpoint modulator, the level of said biomarker in said blood sample, or fraction thereof, is compared with the second reference, which is the level of said biomarker in a reference blood sample, or a fraction thereof, obtained from the patient before start of treatment with said immune checkpoint modulator or the level of said biomarker in blood, or a fraction thereof, of a healthy human population.

In another aspect, the invention provides a method of assessing efficacy of an immune checkpoint modulator in treating a patient diagnosed with a disease, disorder, condition or injury of the CNS, said method comprising determining ex vivo, in a blood sample obtained from the patient, or in a fraction thereof, a biomarker selected from: (a) the level of a monocyte subpopulation (CD14⁺ cells) expressing CCR2 or CD204 or a combination thereof, or CCR2 and a marker selected from igf1, yve1, Stab-1, Siglec1 and Mrc1, or any combination thereof; (b) the ratio of the level of a monocyte subpopulation (CD14⁺ cells) expressing CCR2^{high}CX3CR1^{low} to a monocyte subpopulation (CD14⁺ cells) expressing CCR2^{low}CX3CR1^{high}; (c) the level of a CCR2 agonist selected from CCL2, CCL7, CCL13, CCL8, CCL11 and CCL16; and (d) the level of a CCR2 antagonist selected from CCL24 and CCL26, wherein an equal or increased level of said biomarker (a) to (c) or a decreased level of said biomarker (d) in the blood sample, or a fraction thereof, as compared to a first or a second reference indicates that the immune checkpoint modulator is likely to be efficacious in treating said disease, disorder, condition or injury of the CNS in said patient, and in case the blood sample is obtained from the patient prior to treatment with said immune checkpoint modulator, the level of said biomarker in said blood sample, or fraction thereof, is compared with the first reference, which is the level of said biomarker in blood, or a fraction thereof, of a responder patient population before start of treatment with said immune checkpoint modulator; or in case the blood sample is obtained from the patient after treatment with said immune checkpoint modulator, the level of said biomarker in said blood sample, or fraction thereof, is compared with the second reference, which is the level of said biomarker in a reference blood sample, or a fraction thereof, obtained from the patient before start of treatment with said immune checkpoint modulator or the level of said biomarker in blood, or a fraction thereof, of a healthy human population.

In an additional aspect, the present invention provides a method for excluding a patient diagnosed with a disease, disorder, condition or injury of the CNS from treatment with an immune checkpoint modulator, said method comprising determining ex vivo, in a blood sample obtained from the patient, or in a fraction thereof, a biomarker selected from: (a) the level of a monocyte subpopulation (CD14⁺ cells) expressing CCR2 or CD204 or a combination thereof, or CCR2 and a marker selected from igf1, lyve1, Stab-1, Siglec1 and Mrc1, or any combination thereof; (b) the ratio of the level of a monocyte subpopulation (CD14⁺ cells) expressing CCR2^{high}CX3CR1^{low} to a monocyte subpopulation (CD14⁺ cells) expressing CCR2^{low}CX3CR1^{high}; (c) the level of a CCR2 agonist selected from CCL2, CCL7, CCL13, CCL8, CCL11 and CCL16; and (d) the level of a CCR2 antagonist selected from CCL24 and CCL26, wherein an equal or decreased level of any one of said biomarker (a) to (c) or an increased level of said biomarker (d) in the blood sample, or a fraction thereof, as compared to said first or second reference indicates that the patient is likely to be non-responsive to treatment with said immune checkpoint modulator and is therefore excluded from treatment with said immune checkpoint modulator, and in case the blood sample is obtained from the patient prior to treatment with said immune checkpoint modulator, the level of said biomarker in said blood sample, or fraction thereof, is compared with the first reference, which is the level of said biomarker in blood, or a fraction thereof, of a responder patient population before start of treatment with said immune checkpoint modulator; or in case the blood sample is obtained from the patient after treatment with said immune checkpoint modulator, the level of said biomarker in said blood sample, or fraction thereof, is compared with the second reference, which is the level of said biomarker in a reference blood sample, or a fraction thereof, obtained from the patient before start of treatment with said immune checkpoint modulator or the level of said biomarker in blood, or a fraction thereof, of a healthy human population.

In yet an additional aspect, the present invention provides a method for treating a patient diagnosed with a disease, disorder, condition or injury of the CNS, the method comprising determining ex vivo, in a blood sample obtained from the patient a biomarker selected from: (a) the level of a monocyte subpopulation (CD14⁺ cells) expressing CCR2, CD204 or a combination thereof; or CCR2 and a marker selected from igf1, lyve1, Stab-1, Siglec1 and Mrc1, or any combination thereof; (b) the ratio of the level of a monocyte subpopulation (CD14⁺ cells) expressing CCR2^{high}CX3CR1^{low} to a monocyte subpopulation (CD14⁺ cells) expressing CCR2^{low}CX3CR1^{high}; (c) the level of a CCR2 agonist selected from CCL2, CCL7, CCL13, CCL8, CCL11 and CCL16; and (d) the level of a CCR2 antagonist selected from CCL24 and CCL26, and initiating or continuing administration of an immune checkpoint modulator to said patient if the level in the blood sample, or a fraction thereof, of said biomarker (a) to (c) is equal or increased or the level of the biomarker (d) is decreased as compared to a first or a second reference, wherein in case the blood sample is obtained from the patient prior to treatment with said immune checkpoint modulator, the level of said biomarker in said blood sample, or fraction thereof, is compared with the first reference, which is the level of said biomarker in blood, or a fraction thereof, of a responder patient population before start of treatment with said immune checkpoint modulator; or in case the blood sample is obtained from the patient after treatment with said immune checkpoint modulator, the level of said biomarker in said blood sample, or fraction thereof, is compared with the second reference, which is the level of said biomarker in a reference blood sample, or a fraction thereof, obtained from the patient before start of treatment with said immune checkpoint modulator or the level of said biomarker in blood, or a fraction thereof, of a healthy human population.

In still an additional aspect, the present invention provides a kit for predicting whether a patient diagnosed with a disease, disorder, condition or injury of the CNS is likely to be responsive or non-responsive to treatment with an immune checkpoint modulator, or for assessing the efficacy of an immune checkpoint modulator in treating a patient diagnosed with a disease, disorder, condition or injury of the CNS, said kit comprises reagents useful for determining the patients level of a biomarker selected from: (a) the level of a monocyte subpopulation (CD14⁺ cells) expressing CCR2 or CD204 or a combination thereof, or CCR2 and a marker selected from CD204, igf1, lyve1, Stab-1, Siglec1 and Mrc1, or any combination thereof; (b) the ratio of the level of a monocyte subpopulation (CD14⁺ cells) expressing CCR2^{high}CX3CR1^{low} to a monocyte subpopulation (CD14⁺ cells) expressing CCR2^{low}CX3CR1^{high}; (c) the level of a CCR2 agonist selected from CCL2, CCL7, CCL13, CCL8, CCL11 and CCL16; and (d) the level of a CCR2 antagonist selected from CCL24 and CCL26.

### BRIEF DESCRIPTION OF DRAWINGS

FIGS. 1A-G show that monocyte-derived macrophages uniquely affect disease modification in PD-L1 blockade in DM-hTAU mice. a, Flow cytometry of splenocytes, CD44⁺CD62L^{low} effector memory T (TEM) cells, versus CD44⁺CD62L^{high} central memory T (T_{CM}) cells in DM-hTAU mice, treated with 0.5 mg of anti-PD-L1 (n=10) or IgG (n=11) (one-way ANOVA, Fisher's exact test). b, Flow cytometry of brains from anti-PD-L1-treated mice (n=10), and IgG-treated mice (n=16) analyzed for CD45^{high}CD11b^{high}, pooled from two experiments. c, Quantitation of the number of GFP+CD45^{high} CD11b^{high} cells in anti-PD-L1 (n=4), relative to IgG-treated mice (n=6). d, Sorted CD45^{high}CD11b^{high} from DM-hTAU mice treated with anti-PD-L1, analyzed by single-cell RNASeq. tSNE plot depicting 899 cells. Clusters indicated by color and number. e, Average Unique Molecular Identifier counts for selected genes across the 12 clusters. f, T-maze task, 2 weeks after BM transplant, of WT>WT (n=4), MSR1^{-/-}>WT (n=5), WT>DM-hTAU (n=8) and MSR1^{-/-}>DM-hTAU (n=8) chimeric mice. g, The same mice were treated after the behavioral assessment in m with 1.5 mg of anti-PD-L1 antibody or IgG control antibody, and were tested again 1 month later for their performance in T-maze; non chimeric IgG-treated DM-hTAU littermates were used as additional controls. Improved performance of WT>DM-hTAU treated with anti-PD-L1 (n=5) versus IgG-treated WT>DM-hTAU (n=3) and IgG-treated non chimeric DM-hTAU mice (n=6). MSR1^{-/-}>DM-hTAU mice failed to show beneficial effect following treatment with anti-PD-L1 (n=5), performing similarly to MSR1^{-/-}>DM-hTAU treated with IgG (n=3). In all panels, error bars represent mean±s.e.m.; *P<0.05, **P<0.01, ***P<0.001 (one-way ANOVA and Fisher's exact test).

FIG. 2 shows that PD-L1 inhibition modifies the immune landscape of blood derived from 5×FAD mice. Eight-month old AD or WT mice were treated or not intraperitoneally with either 1.5 mg of αPD-L1 or IgG2b and euthanised 3 (3D) or 5 (5D) days after the administration. Peripheral blood mononuclear cells were isolated and stained for subsequent mass cytometric analysis through Cytofkit (R/Bioconductor). Frequency of CCR2+ myeloid cells when gated on CD45. Results are representative of four independent experiments combined (n=3-4 animals per group). Data are expressed as mean±s.e.m. Means between groups were compared with one-way analysis of variance followed by a Tukey's post-hoc test. Statistical significance levels were set as follows: treatment versus untreated WT: ##p<0.01; αPD-L1 versus IgG2b: *p<0.05, **p<0.01.

FIGS. 3A-G show that MC21 treatment reduces monocyte populations in the blood without behavioral phenotype. Anti-CCR2 antibody MC21 was intraperitoneally (i.p.) injected every 4 days to total of 4 injections. Control animals were not treated. Three days after the 4^{th} injection blood was collected and analyzed by flow cytometry. a. Flow cytometry analyses of Ly6G⁻CD115⁺ myeloid cells (Student's t-test: t_{(two-taled)}=²0.256, df=14, *p=0.0406) and of b. Ly6C⁺ myeloid populations (Student's t-test: Ly6C^{hi} t_{(two-tailed)}=3.764, df=14, *p=0.0021; Ly6C^{med}t_{(two-taled)}=2.442, df=14, *p=0.0285) in control and MC21-injected groups. c. Flow cytometry analyses of CD4 T cells and d. memory CD4 T cell populations. n=8 mice per group. MC21 was i.p. injected 4-5 times and during the 4 days after the last injection the cognitive behavior of the animals was assessed by e. Percent novel arm exploration time (out of all 3 arms) as measured in the T-maze. f. Percent spontaneous alternation as calculated in the Y-maze. g. Percent novel object exploration time (out of the 2 objects). n=6 mice per group. Data are presented as mean±s.e.m. *p<0.05.

FIGS. 4A-F show that MC21 treatment abrogates the beneficial effect of PD-L1 blockade. a. MC21 was i.p. injected 3 days prior (Day −3) to αPD-L1 (Day 0), and then again on days 1, 5 and 9. One month after αPD-L1 treatment the cognitive behavior of the animals was assessed by T-maze, spontaneous alternation test in Y-maze and novel object recognition. Subsequently the mice' brains were extracted and Aggregated Tau levels in cortices were measured. b. Percent novel arm exploration time (out of all 3 arms) as measured in the T-maze (One-way ANOVA F_{(4,56)}=9.068, ***p<0.0001). c. Percent spontaneous alternation as calculated in the Y-maze (One-way ANOVA F_{(4,55)}=19.73, ***p<0.0001). d. Percent novel object exploration time (out of the 2 objects. One-way ANOVA F_{(4,52)}=12.48, ***p<0.0001). n=9-18 mice per group. Data are presented as mean±s.e.m. Post-hoc Tukey's multiple comparisons between DM-hTAU groups to the WT: *p<0.05, **p<0.01, ***p<0.001. Post-hoc Tukey's multiple comparisons between the DM-hTAU groups #p<0.05, ##p<0.01, ###p<0.001. e. Aggregated Tau protein in cortices of treated DM-hTAU mice in comparison for the control IgG-treated and WT groups (One-way ANOVA F_{(4,28)}=7.409, ***p=0.0003. Post-hoc Fisher's LSD multiple comparisons: *p<0.05, **p<0.01, ***p<0.001). n=8-6 mice per group. Data are presented as mean±s.e.m. f. Correlation between the measured Aggregated Tau protein in cortices versus cognitive behavior as assessed by T-maze.

FIG. 5 shows that blocking CCR2 abolishes the αPD-L1-induced upregulation of CCR2+ myeloid cells in blood. Three days following αPD-L1 treatment the blood of the mice was analyzed by CyTOF. Frequency of CCR2⁺ myeloid cells presented as a ratio to IgG (One-way ANOVA F_{(3,19)}=7.854, **p<0.01, ***p<0.001). N=5-6 mice per group. Data are presented as mean±s.e.m.

### DETAILED DESCRIPTION OF THE INVENTION

It has been known for some time now that revitalizing systemic immunity using antibodies that block either Programmed cell death protein 1 (PD-1) or its ligand, PD-L1, could modify brain pathology and restore cognitive performance in a mouse model of tauopathy (DM-hTAU), in addition to its effect in a β-amyloid Alzheimer's disease (AD) model, and that this effect is mediated, at least in part, via recruitment of monocyte-derived macrophages^{8,9,12} It was also demonstrated that a systemic IFN-γ-dependent immune response was evoked by using neutralizing antibodies for PD-1 and PD-L1 and T-cell immunoglobulin and mucin-domain containing-3 (TIM-3) and that this IFN-γ-dependent immune response was needed in order to mobilize immune cells to the CNS. When induced in animals with established AD pathology, treatment with these neutralizing antibodies resulted in an immunological response that cleared of cerebral amyloid-β plaques and improved cognitive performance. Thus, using neutralizing antibodies for three different immune checkpoint members resulted in an IFN-γ-dependent immune response that reversed the disease state (WO 2015/136541; WO 2017/009829; WO 2018/047178).

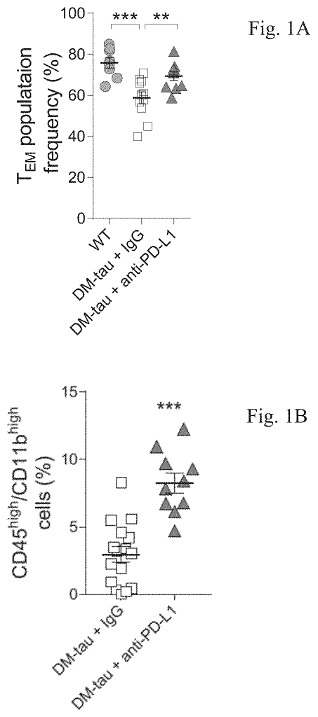

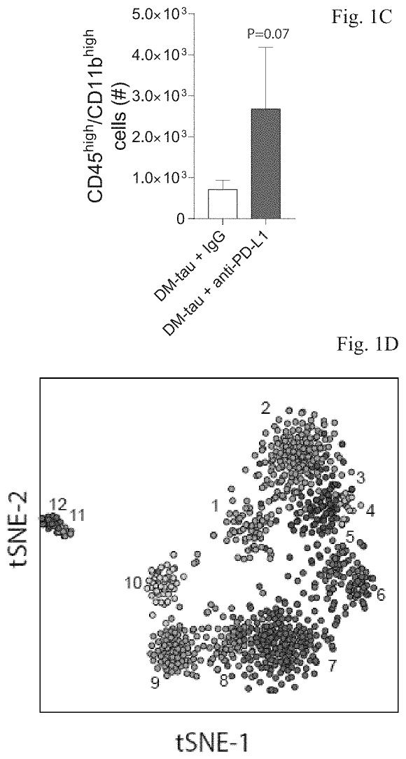

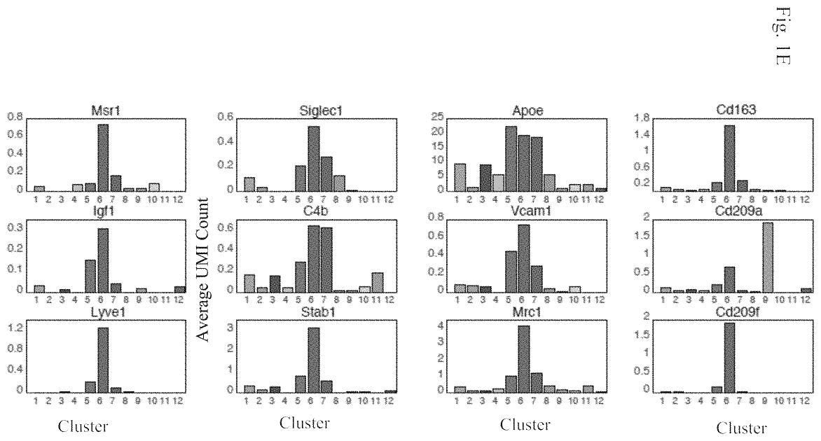

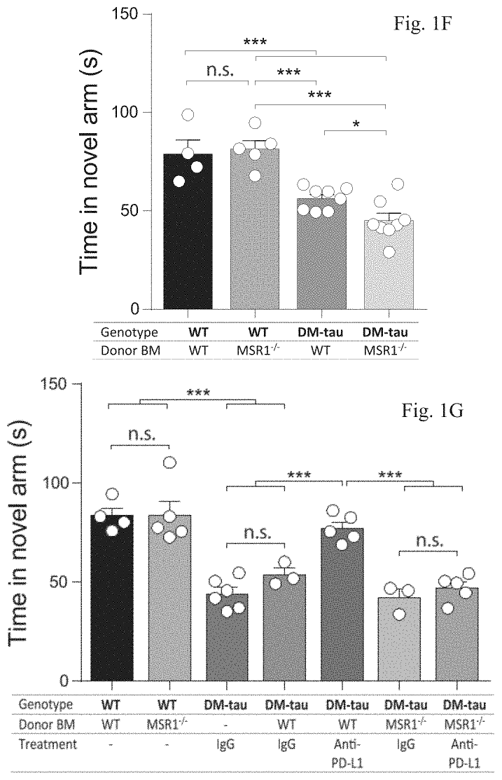

The present invention is based on the findings disclosed in Examples 1-3 that blockade of the PD-1/PD-L1 axis in a mouse model of Alzheimer's disease results in increase of a specific monocyte subpopulation (MSR-1⁺CCR2⁺ myeloid cell population) in the blood and enhances recruitment of these cells to the brain parenchyma. It was further found by the present inventors that the infiltrating monocyte-derived macrophages are heterogeneous. Analysis of differential genes in each cluster highlighted a unique signature manifested by expression of several molecules that could potentially mediate an important function in disease modification (FIGS. 1d,e). One such uniquely expressed molecule is the macrophage scavenger receptor 1 (Msr1) (also known as SRA1, SCARA1, or CD204), an important phagocytic receptor required for engulfment of misfolded and aggregated proteins 17,18. Notably, these macrophages expressed other relevant functional molecules, among which are the insulin-like growth factor-1 (igf1) that was previously reported to enhance neurogenesis in the aged brain¹⁹, lymphatic endothelium-specific hyaluronan receptor (lyve1) and the scavenger receptor stabilin-1 (Stab-1) (FIG. 1e), both of which are markers of anti-inflammatory macrophages, associated with wound healing and lymphogenesis²⁰ Additional genes, found here to be uniquely expressed by infiltrating monocyte-derived macrophages, are CCR2 and scavenger receptors such as the sialic acid binding Ig like lectin 1 (Siglec1) and the mannose receptor C-type (Mrc1) (FIG. 1e). Importantly, it was found that blockade of the PD-1/PD-L1 pathway using a neutralizing anti-PD-L1 antibody resulted in an increase in a subpopulation characterized by expression of macrophage scavenger receptor 1 (CD204), and optionally also CX3CR1, Ki67, IBA-1, and Sca. Furthermore, it is expected that insulin-like growth factor-1 (igf1), lymphatic endothelium-specific hyaluronan receptor (lyve1), scavenger receptor stabilin-1 (Stab-1), sialic acid binding Ig like lectin 1 (Siglec1) and mannose receptor C-type (Mrc1) are also expressed on the cells of this subpopulation.

An additional important discovery is that antibodies that block either PD-1 or its ligand, PD-L1, failed to modify brain pathology and restore cognitive performance in MSR1-deficient chimeric DM-hTAU mice, unlike the situation in MSR-1+DM-hTAU mice, for which the antibodies were efficacious (Example 2). This indicates that monocytes expressing, or being capable of expressing, at least the MSR-1 marker can serve as a prognostic marker for the response of a patient diagnosed with a disease, disorder, condition or injury of the Central Nervous System (CNS) to treatment with an immune checkpoint modulator.

CCR2 is a chemokine receptor expressed mainly by monocytes, and was shown to play a critical role for monocyte migration from the bone marrow to the blood and for recruitment of inflammatory monocytes into the injured/diseased brain²¹⁻²³. It was further found herein that blockade of CCR2 in a mouse model of tau pathology abrogates the beneficial effect of PD-L1 blockade (Example 5), and abolishes the anti-PD-L1 antibody induced upregulation of CCR2+ myeloid cells in blood (Example 6). This indicates that monocytes expressing, or being capable of expressing, the CCR2 marker alone or in combination with other markers mentioned above can serve as a prognostic marker for the response of a patient diagnosed with a disease, disorder, condition or injury of the CNS to treatment with an immune checkpoint modulator.

Since the activity of CCR2 is critical for monocyte migration into the CNS, CCR2 agonists or antagonists can also serve as prognostic markers for the response of a patient diagnosed with a disease, disorder, condition or injury of the CNS to treatment with an immune checkpoint modulator, i.e. a monocyte population expressing low levels of CCR2 is functionally equivalent, in terms of serving as a prognostic marker, to a high blood level of a soluble CCR2 antagonist or a low level of a soluble CCR2 agonist. The blockade of CCR2 was achieved herein by using a neutralizing anti-CCR2 antibody as a non-limiting example; however, it exemplifies that the level of any CCR2 agonist or antagonist can be used as a biomarker for the effect of PD-1/PD-L1 blockade treatment, such as eotaxin-3 (aka Chemokine (C—C motif) ligand 26 (CCL26), Macrophage inflammatory protein 4-alpha (MIP-4-alpha), Thymic stroma chemokine-1 (TSC-1) and IMAC). (Bachelerie F, Ben-Baruch A, Charo I F, Combadiere C, Farber J M, Forster R, Graham G J, Hills R, Horuk R, Locati M, Luster A D, Mantovani A, Matsushima K, Monaghan A E, Moschovakis G L, Murphy P M, Nibbs R J B, Nomiyama H, Oppenheim J J, Power C A, Proudfoot A E I, Rosenkilde M M, Rot A, Sozzani S, Thelen M, Uddin M, Yoshie O, Zlotnik A. Chemokine receptors (version 2019.5) in the IUPHAR BPS Guide to Pharmacology Database. IUPHAR/BPS Guide to Pharmacology CITE. 2019; 2019(5).)

Furthermore, the ratio of a monocyte subpopulation expressing CCR2^{high}CX3CR1^{low} to a monocyte subpopulation expressing CCR2^{low}CX3CR1^{high}, can also serve as a prognostic marker since the CCR2 antagonist eotaxin-3 is also an agonist of CX3CR1.

Generally, the immune response which is mounted following immune checkpoint blockade is largely associated with IFN-γ or T cells which produce IFN-γ. As such, the present invention is useful for predicting whether a patient diagnosed with a disease, disorder, condition or injury of the CNS is likely to be responsive or non-responsive to treatment with an immune checkpoint modulator of any immune checkpoint member that suppresses an IFN-γ-dependent immune response.

In view these findings and underlying facts, in one aspect, the present invention provides a method for predicting whether a patient diagnosed with a disease, disorder, condition or injury of the CNS is likely to be responsive or non-responsive to treatment with an immune checkpoint modulator, said method comprising determining ex vivo, in a blood sample obtained from the patient, or in a fraction thereof, a biomarker selected from: (a) the level of a monocyte subpopulation (CD14⁺ cells) expressing C—C chemokine receptor type 2 (CCR2, a.k.a. CD192) or macrophage scavenger receptor 1 (MSR-1, a.k.a. SRA1, SCARA1 and CD204) or a combination thereof, or CCR2 and a marker selected from insulin-like growth factor-1 (igf1), lymphatic endothelium-specific hyaluronan receptor (lyve1), scavenger receptor stabilin-1 (Stab-1), sialic acid binding Ig like lectin 1 (Siglec1) and mannose receptor C-type (Mrc1), or any combination thereof; (b) the ratio of the level of a monocyte subpopulation (CD14⁺ cells) expressing CCR2^{high}CX3CR1^{low} to a monocyte subpopulation (CD14⁺ cells) expressing CCR2^{low}CX3CR1^{high}; (c) the level of a CCR2 agonist selected from CCL2, CCL7, CCL13, CCL8, CCL11 and CCL16; and (d) the level of a CCR2 antagonist selected from CCL24 and CCL26, wherein an equal or increased level of said biomarker (a) to (c) or a decreased level of said biomarker (d) in the blood sample, or a fraction thereof, as compared to a first or a second reference indicates that the patient is likely to be responsive to treatment with said immune checkpoint modulator, and an equal or decreased level of any one of said biomarker (a) to (c) or an increased level of said biomarker (d) in the blood sample, or a fraction thereof, as compared to said first or second reference indicates that the patient is likely to be non-responsive to treatment with said immune checkpoint modulator, and in case the blood sample is obtained from the patient prior to treatment with said immune checkpoint modulator, the level of said biomarker in said blood sample, or fraction thereof, is compared with the first reference, which is the level of said biomarker in blood, or a fraction thereof, of a responder patient population before start of treatment with said immune checkpoint modulator; or in case the blood sample is obtained from the patient after treatment with said immune checkpoint modulator, the level of said biomarker in said blood sample, or fraction thereof, is compared with the second reference, which is the level of said biomarker in a reference blood sample, or a fraction thereof, obtained from the patient before start of treatment with said immune checkpoint modulator or the level of said biomarker in blood, or a fraction thereof, of a healthy human population.

In another aspect, the invention provides a method of assessing efficacy of an immune checkpoint modulator in treating a patient diagnosed with a disease, disorder, condition or injury of the CNS, said method comprising determining ex vivo, in a blood sample obtained from the patient, or in a fraction thereof, a biomarker selected from: (a) the level of a monocyte subpopulation (CD14⁺ cells) expressing CCR2 or CD204 or a combination thereof, or CCR2 and a marker selected from igf1, yve1, Stab-1, Siglec1 and Mrc1, or any combination thereof; (b) the ratio of the level of a monocyte subpopulation (CD14⁺ cells) expressing CCR2^{high}CX3CR1^{low} to a monocyte subpopulation (CD14⁺ cells) expressing CCR2^{low}CX3CR1^{high}; (c) the level of a CCR2 agonist selected from CCL2, CCL7, CCL13, CCL8, CCL11 and CCL16; and (d) the level of a CCR2 antagonist selected from CCL24 and CCL26, wherein an equal or increased level of said biomarker (a) to (c) or a decreased level of said biomarker (d) in the blood sample, or a fraction thereof, as compared to a first or a second reference indicates that the immune checkpoint modulator is likely to be efficacious in treating said disease, disorder, condition or injury of the CNS in said patient, and in case the blood sample is obtained from the patient prior to treatment with said immune checkpoint modulator, the level of said biomarker in said blood sample, or fraction thereof, is compared with the first reference, which is the level of said biomarker in blood, or a fraction thereof, of a responder patient population before start of treatment with said immune checkpoint modulator; or in case the blood sample is obtained from the patient after treatment with said immune checkpoint modulator, the level of said biomarker in said blood sample, or fraction thereof, is compared with the second reference, which is the level of said biomarker in a reference blood sample, or a fraction thereof, obtained from the patient before start of treatment with said immune checkpoint modulator or the level of said biomarker in blood, or a fraction thereof, of a healthy human population.

In an additional aspect, the present invention provides a method for excluding a patient diagnosed with a disease, disorder, condition or injury of the CNS from treatment with an immune checkpoint modulator, said method comprising determining ex vivo, in a blood sample obtained from the patient, or in a fraction thereof, a biomarker selected from: (a) the level of a monocyte subpopulation (CD14⁺ cells) expressing CCR2 or CD204 or a combination thereof, or CCR2 and a marker selected from igf1, lyve1, Stab-1, Siglec1 and Mrc1, or any combination thereof; (b) the ratio of the level of a monocyte subpopulation (CD14⁺ cells) expressing CCR2^{high}CX3CR1^{low} to a monocyte subpopulation (CD14⁺ cells) expressing CCR2^{low}CX3CR1^{high}; (c) the level of a CCR2 agonist selected from CCL2, CCL7, CCL13, CCL8, CCL11 and CCL16; and (d) the level of a CCR2 antagonist selected from CCL24 and CCL26, wherein an equal or decreased level of any one of said biomarker (a) to (c) or an increased level of said biomarker (d) in the blood sample, or a fraction thereof, as compared to said first or second reference indicates that the patient is likely to be non-responsive to treatment with said immune checkpoint modulator and is therefore excluded from treatment with said immune checkpoint modulator, and in case the blood sample is obtained from the patient prior to treatment with said immune checkpoint modulator, the level of said biomarker in said blood sample, or fraction thereof, is compared with the first reference, which is the level of said biomarker in blood, or a fraction thereof, of a responder patient population before start of treatment with said immune checkpoint modulator; or in case the blood sample is obtained from the patient after treatment with said immune checkpoint modulator, the level of said biomarker in said blood sample, or fraction thereof, is compared with the second reference, which is the level of said biomarker in a reference blood sample, or a fraction thereof, obtained from the patient before start of treatment with said immune checkpoint modulator or the level of said biomarker in blood, or a fraction thereof, of a healthy human population.

In yet an additional aspect, the present invention provides a method for treating a patient diagnosed with a disease, disorder, condition or injury of the CNS, the method comprising determining ex vivo, in a blood sample obtained from the patient a biomarker selected from: (a) the level of a monocyte subpopulation (CD14⁺ cells) expressing CCR2, CD204 or a combination thereof; or CCR2 and a marker selected from igf1, lyve1, Stab-1, Siglec1 and Mrc1, or any combination thereof; (b) the ratio of the level of a monocyte subpopulation (CD14⁺ cells) expressing CCR2^{high}CX3CR1^{low} to a monocyte subpopulation (CD14⁺ cells) expressing CCR2^{low}CX3CR1^{high}; (c) the level of a CCR2 agonist selected from CCL2, CCL7, CCL13, CCL8, CCL11 and CCL16; and (d) the level of a CCR2 antagonist selected from CCL24 and CCL26, and initiating or continuing administration of an immune checkpoint modulator to said patient if the level in the blood sample, or a fraction thereof, of said biomarker (a) to (c) is equal or increased or the level of the biomarker (d) is decreased as compared to a first or a second reference, wherein in case the blood sample is obtained from the patient prior to treatment with said immune checkpoint modulator, the level of said biomarker in said blood sample, or fraction thereof, is compared with the first reference, which is the level of said biomarker in blood, or a fraction thereof, of a responder patient population before start of treatment with said immune checkpoint modulator; or in case the blood sample is obtained from the patient after treatment with said immune checkpoint modulator, the level of said biomarker in said blood sample, or fraction thereof, is compared with the second reference, which is the level of said biomarker in a reference blood sample, or a fraction thereof, obtained from the patient before start of treatment with said immune checkpoint modulator or the level of said biomarker in blood, or a fraction thereof, of a healthy human population.

Below are disclosed non-limiting embodiments of any one of the above aspects.

In certain embodiments, the immune checkpoint modulator is selected from an agonistic or antagonistic: (i) antibody, such as a humanized antibody; a human antibody; a functional fragment of an antibody; a single-domain antibody, such as a Nanobody; a recombinant antibody; and a single chain variable fragment (ScFv); (ii) antibody mimetic, such as an affibody molecule; an affilin; an affimer; an affitin; an alphabody; an anticalin; an avimer; a DARPin; a fynomer; a Kunitz domain peptide; and a monobody; (iii) aptamer; and (iv) a small molecule.

As stated above, the present invention is useful for predicting whether a patient diagnosed with a disease, disorder, condition or injury of the CNS is likely to be responsive or non-responsive to treatment with an immune checkpoint modulator of any immune checkpoint member that suppresses an IFN-γ-dependent immune response. With this in mind, the following non-limiting examples of immune checkpoint members (in addition to PD-1/PD-L1 and TIM-3) are also known to suppress an IFN-γ-dependent immune response.

Importantly, some immune checkpoint molecules can be considered as “off switches” on the immune response, their blockade activates the immune system, and thus these are referred to as “negative regulators”. Other immune checkpoint molecules can be considered as “on switches” on the immune response, their stimulation activates the immune system, and thus these are referred to as “positive regulators”. Many of these molecules are members of the B7 family, and they act as rheostats that control the threshold for whether a given T-cell receptor (TCR) interaction leads to activation and/or anergy. Targeting either negative regulators or positive regulators checkpoints leads to an IFN-γ-dependent immune response.

Negative Regulators:

CTLA4, the first immune checkpoint receptor to be clinically targeted, is expressed exclusively on T cells where it primarily regulates the amplitude of the early stages of T cell activation. Primarily, CTLA4 counteracts the activity of the T cell co-stimulatory receptor, CD28 (Pardoll, 2012 The blockade of immune checkpoints in cancer immunotherapy. Nat. Rev. Cancer 12, 252-264). In 2001, Paradis et al. were the first to show that the anti-tumor activity of anti-CTLA-4 is mediated through its induction of IFN-7 (Paradis et al., 2001 The anti-tumor activity of anti-CTLA-4 is mediated through its induction of IFN gamma. Cancer Immunol. Immunother. 50, 125-133). Since then, this mechanism has been substantiated by other groups, specifically showing that: (1) CTLA-4 blockade increases IFN-γ-producing CD4 ICOS-high cells (Liakou et al., 2008 CTLA-4 blockade increases IFN-γ-producing CD4 ICOS hi cells to shift the ratio of effector to regulatory T cells in cancer patients), and (2) loss of IFN-7 pathway genes confers resistance to anti-CTLA-4 Therapy in cancer (Gao et al., 2016 Loss of IFN-7 Pathway Genes in Tumor Cells as a Mechanism of Resistance to Anti-CTLA-4 Therapy. Cell 167, 397-404.e9).

LAG-3 provides an inhibitory signal to activated effector T cells and augments the suppressive activity of Treg cells. MHC class II is the only known ligand for LAG3 and LAG-3/MHC class II interaction down-regulates T-cell mediated immune responses. LAG-3 has been shown to negatively regulate cellular proliferation, activation, and homeostasis of T cells, in a similar fashion to CTLA-4 and PD-1. In particular, LAG-3 is important for the suppressive functions of CD4⁺ Tregs in autoimmune responses, and for maintaining tolerance to self and tumor antigens via dampening the activity of antigen-specific CD8⁺ T cells. Similar to PD-1, there is a dramatic increase of the percentage of LAG-3⁺CD8⁺ T cells and of LAG-3⁺CD4⁺ T cells present in tumor infiltrating lymphocytes as compared to controls. The increased expression of PD-1 and LAG-3 render CD8⁺ T cells incapable of mounting an effective anti-tumor immune response. Furthermore, studies have shown a synergistic role of PD-1 and LAG-3 in suppressing T cell functions. Thus, taken together, neutralizing LAG-3 activity should result in an IFN-γ-dependent immune response that reversed the disease state.

V-domain immunoglobulin (Ig)-containing suppressor of T-cell activation (VISTA) is predominantly expressed on hematopoietic cells, and in multiple murine cancer models is found at particularly high levels on myeloid cells and Foxp3+CD4+ regulatory cells (Lines et al., 2014 VISTA is a novel broad-spectrum negative checkpoint regulator for cancer immunotherapy. Cancer Immunol. Res. 2, 510-517). Similar to some members of the B7-CD28 family (e.g., PD-L1), T cells both express and respond to VISTA. VISTA blockade impairs the suppressive function of Foxp3+CD4+ regulatory T cells, which is one mechanism by which it was suggested to evoke an IFN-g response (Le Mercier et al., 2014 VISTA Regulates the Development of Protective Antitumor Immunity. Cancer Res. 74, 1933-1944). Indeed, following VISTA blockade there is increased number of IFN-γ-producing cells and anti-tumor immunity is augmented (Le Mercier et al., 2014, supra).

Within the signaling pathways that govern NK cell activity, the killer cell immunoglobulin-like receptor (KTR) family is a dominant group of negative regulators. KIR receptors bind to the self-MHC class I ligands (HLA-A, -B, -C) and upon ligation transmit signals that abrogate the effects of activating receptors. Preventing HLA ligation to KIRs with an anti-KIR mAb has been shown to increase IFN-γ secretion, and tumor cell lysis as well as increasing overall survival in murine cancer models (Koh et al., 2001 Augmentation of antitumor effects by NK cell inhibitory receptor blockade in vitro and in vivo. Blood 97, 3132-313).

A2A adenosine receptor (A2AR), and the adenosine generating enzyme, CD73 are expressed by many immune cell populations. Stimulation of A2AR generally provides an immunosuppressive signal that inhibits activities of T cells (proliferation, cytokine production, cytotoxicity), NK cells (cytotoxicity), NKT cells (cytokine production, CD40L upregulation), macrophages/dendritic cells (antigen presentation, cytokine production), and neutrophils (oxidative burst) (Ohta, 2016). Specifically, A2AR stimulation in effector T cells (Teff) blocks T cell receptor signaling and impairs IFN-γ production, while A2AR or CD73 blockade can induce an IFN-γ-dependent immune response (Allard et al., 2013; Leone et al., 2015).

Positive Regulators

B7 homolog 3 (B7-H3) was first identified in 2001 as a costimulatory molecule for T cell activation and IFN-gamma production (Chapoval et al., 2001 B7-H3: a costimulatory molecule for T cell activation and IFN-gamma production. Nat. Immunol. 2, 269-274). B7-H3 costimulates proliferation of both CD4+ and CD8+ T cells, enhances the induction of cytotoxic T cells and selectively stimulates interferon gamma production in the presence of T cell receptor signaling.

The inducible co-stimulatory receptor (ICOS) shares much homology with CD28, yet key differences in signaling mechanisms and unique expression patterns of ICOS ligand suggest non-redundant functions. Similar to CTLA-4, ICOS is induced following T cell activation (Sharpe and Freeman, 2002 The B7-CD28 Superfamily. Nat. Rev. Immunol. 2, 116-126). The ICOS receptor is engaged by ICOSL, another member of the B7 family. ICOSL is expressed in APCs (B cells, macrophages, dendritic cells) and can be induced by inflammatory cytokines in non-hematopoietic cells including endothelial cells and epithelial cells. In vitro, ICOS co-stimulation of peripheral T cells from patients with active SLE results in greatly enhanced IFN-γ production relative to normal controls (Kawamoto et al., 2006 Expression and function of inducible co-stimulator in patients with systemic lupus erythematosus: possible involvement in excessive interferon-gamma and anti-double-stranded DNA antibody production. Arthritis Res. Ther. 8, R62). In tuberculosis patients, ICOS expression significantly correlates with IFN-gamma production, and ICOS ligation augments Ag-specific secretion of the Th1 cytokine IFN-gamma from responsive individuals (Quiroga et al., 2006 Inducible costimulator: a modulator of IFN-gamma production in human tuberculosis. J. Immunol. 176, 5965-5974).

CD137 (4-1BB/TNFRSF9) was the first TNFRSF member to be identified as a possible immunotherapy target (Melero et al., 1997 Monoclonal antibodies against the 4-1BB T-cell activation molecule eradicate established tumors. Nat. Med. 3, 682-685). The family includes 28 other receptors that are implicated in cellular activation and survival and are being considered or tested as immunotherapeutic targets, including CD134 (OX40/TNFRSF4), CD40 (TNFRSF5), CD27 (TNFRSF7), CD270 (HVEM/TNFRSF14), and CD357 (GITR/TNFRSF18). In T cells and NK cells, CD137 activation induces proliferation and production of interferon gamma, and the CD137-mediated anti-tumor response was characterized to be dependent on IFN-γ for regulating the infiltration of antigen-specific T cells into the tumor (Makkouk et al., 2016 Rationale for anti-CD137 cancer immunotherapy. Eur. J. Cancer 54, 112-119).

OX40, also known as CD134 or TNFRSF4, is a co-stimulatory molecule expressed primarily by activated T cells, but also expressed on natural killer T (NKT) cells and NKs. In NK cells, OX40 ligation appears to induce an activating signal and IFN-γ production (Liu et al., 2008 Plasmacytoid dendritic cells induce NK cell-dependent, tumor antigen-specific T cell cross-priming and tumor regression in mice. J. Clin. Invest. 118, 1165-1175). In addition, OX40 co-stimulation has been reported to enhance the ability of T cells to respond productively to lower affinity antigens and OX40 ligation can enhance IFN-γ production by T cells in response to TCR stimulation (Linch et al., 2015 OX40 Agonists and Combination Immunotherapy: Putting the Pedal to the Metal. Front. Oncol. 5, 34). Furthermore, OX40 triggering appears to be antagonistic for FoxP3 induction in antigen-responding naive CD4⁺ T cells, effectively suppressing the generation of iTreg (Vu et al., 2007 OX40 costimulation turns off Foxp3⁺ Tregs. Blood 110, 2501-2510).

CD27 is another TNFR family member that differs from OX40 in that its expression is constitutive upon different sets of effector T cells. When anti-CD27 agonist is combined with anti-PD-L1, additive effects upon proliferation and synergistic increases in IFN-g expression are observed (Buchan et al., 2015 OX40- and CD27-Mediated Costimulation Synergizes with Anti-PD-L1 Blockade by Forcing Exhausted CD8⁺ T Cells To Exit Quiescence. J. Immunol. 194(1):125-133).

In certain embodiments, the immune checkpoint modulator, including those listed in items (i) to (iv) above, targets or modulates activity of an immune checkpoint selected from PD1-PDL1, PD1-PDL2, CD28-CD80, CD28-CD86, CTLA4-CD80, CTLA4-CD86, ICOS-B7RP1, B7H3, B7H4, B7H7, B7-CD28-like molecule, BTLA-HVEM, KIR-MHC class I or II, LAG3-MHC class I or II, CD137-CD137L, OX40-OX40L, CD27-CD70, CD40L-CD40, TIM3-GAL9, V-domain Ig suppressor of T cell activation (VISTA), STimulator of INterferon Genes (STING), T cell immunoglobulin and immunoreceptor tyrosine-based inhibitory motif domain (TIGIT), A2aR-Adenosine and indoleamine-2,3-dioxygenase (IDO)-L-tryptophan.

In certain embodiments, the immune checkpoint modulator is selected from: (i) an antibody selected from: (a) anti-PD-L1 antibody; (b) anti-PD-1 antibody; (c) anti-TIM-3 antibody; (d) anti-ICOS antibody; (e) anti-PD-L2 antibody; (f) anti-CTLA-4 antibody; (g) anti-B7RP1 antibody; (h) anti-CD80 antibody; (i) anti-CD86 antibody; (j) anti-B7-H3 antibody; (k) anti-B7-H4 antibody; (1) anti-BTLA antibody; (m) anti-HVEM antibody; (n) anti-CD137 antibody; (o) anti-CD137L antibody; (p) anti-CD-27 antibody; (q) anti-CD70 antibody; (r) anti-CD40 antibody; (s) anti-CD40L antibody; (t) anti-OX40 antibody; (u) anti-OX40L antibody; (v) anti-killer-cell immunoglobulin-like receptor (KIR) antibody; (w) anti-LAG-3 antibody; (x) anti-CD47 antibody; (y) anti-VEGF-A antibody; (z) anti-CD25 antibody; (aa) anti-GITR antibody; (bb) anti-CCR4 antibody; (cc) anti-4-1BB antibody; and (dd) any combination of (a) to (cc); (ii) any combination of (a) to (cc) in combination with an adjuvant; (iii) a small molecule selected from: (a) a p300 inhibitor; (b) Sunitinib; (c) Polyoxometalate-1 (POM-1); (d) α,β-methyleneadenosine 5′-diphosphate (APCP); (e) arsenic trioxide (As2O3); (f) GX15-070 (Obatoclax); (g) a retinoic acid antagonist; (h) an SIRPα (CD47) antagonist; (i) a CCR4 antagonist; (j) an adenosine receptor antagonist; (k) an adenosine A1 receptor antagonist; (1) an adenosine A2a receptor antagonist; (m) an adenosine A2b receptor antagonist; (n) an A3 receptor antagonist; (o) an antagonist of indoleamine-2,3-dioxygenase; and (p) an HIF-1 regulator; (iv) any combination of (iii) (a-p) and (i) (a-cc); (v) a protein selected from: (a) Neem leaf glycoprotein (NLGP); and (b) sCTLA-4; (vi) a silencing molecule selected from miR-126 antisense and anti-galectin-1 (Gal-1); (vii) OK-432; (viii) a combination of IL-12 and anti-CTLA-4; (ix) an antibiotic agent; and (x) any combination of (i) to (ix).

In certain embodiments, the antibody used as an immune checkpoint modulator in any one of the above embodiments is an anti-PD-L1 antibody.

In certain embodiments, the antibody used as an immune checkpoint modulator in any one of the above embodiments is an anti-PD-1 antibody.

In certain embodiments, if the immune checkpoint target is a negative immune checkpoint, such as PD-1, then the antibody modulator is an antagonistic antibody.

In certain embodiments, if the immune checkpoint target is a positive immune checkpoint, such as OX40, then the antibody modulator is an agonistic antibody.

In certain embodiments, the anti-PD-L1 antibody used as an immune checkpoint modulator in any one of the above embodiments is an antagonistic anti-PD-1 antibody.

In certain embodiments the anti-PD-1 antibody used as an immune checkpoint modulator in any one of the above embodiments is an antagonistic anti-PD-1 antibody.

In certain embodiments the anti-Siglec-3 antibody used as an immune checkpoint modulator in any one of the above embodiments is an antagonistic anti-Siglec-3 antibody.

In certain embodiments, the anti-TIM3 antibody used as an immune checkpoint modulator in any one of the above embodiments is an antagonistic anti-TIM3 antibody.

In certain embodiments, the anti-ICOS antibody used as an immune checkpoint modulator in any one of the above embodiments is an antagonistic anti-ICOS antibody.

In certain embodiments, the anti-ICOS antibody used as an immune checkpoint modulator in any one of the above embodiments is an agonistic anti-ICOS antibody.

In certain embodiments, the anti-PD-L2 antibody used as an immune checkpoint modulator in any one of the above embodiments is an antagonistic anti-PD-L2 antibody.

In certain embodiments, the anti-CTLA-4 antibody used as an immune checkpoint modulator in any one of the above embodiments is an antagonistic anti-CTLA-4 antibody.

In certain embodiments, the cells of said monocyte cell subpopulation of (a) to (c) in any one of the above embodiments further express a marker selected from CX3CR1, Ki67, IBA-1, and Sca, or any combination thereof.

In certain embodiments, the increased level of the biomarker is increased by a statistically significant difference as compared with the reference. Alternatively, the increased level of the biomarker is increased by 10%, 15%, 20%, 25%, 30%, 35%, 40%, 45%, 50%, 55%, 60%, 65%, 70%, 75%, 80%, 85%, 90%, 95%, 100%, 3-fold, 4-fold, 5-fold, 6-fold, 7-fold, 8-fold, 9-fold, 10 fold, or more as compared with the reference concentration.

In certain embodiments, the decreased level of the biomarker is lower than the reference by a statistically significant difference. Alternatively, the decreased level of the biomarker means that the concentration is 10%, 15%, 20%, 25%, 30%, 35%, 40%, 45%, 50%, 55%, 60%, 65%, 70%, 75%, 80%, 85%, or 90% as compared with the reference concentration.

In certain embodiments, the disease, disorder or condition in any one of the above embodiments is selected from a neurodegenerative disease selected from Alzheimer's disease, a taupathy, amyotrophic lateral sclerosis, Parkinson's disease and Huntington's disease; primary progressive multiple sclerosis; secondary progressive multiple sclerosis; corticobasal degeneration; Rett syndrome; a retinal degeneration disorder selected from age-related macular degeneration and retinitis pigmentosa; anterior ischemic optic neuropathy; glaucoma; uveitis; depression; trauma-associated stress or post-traumatic stress disorder; frontotemporal dementia; Lewy body dementias; mild cognitive impairments; posterior cortical atrophy; primary progressive aphasia; progressive supranuclear palsy; mild cognitive impairment; and aged-related dementia.

A tauopathy is any of the following diseases: argyrophilic grain disease, chronic traumatic encephalopathy, corticobasal degeneration, dementia pugilistica, frontotemporal dementia, frontotemporal lobar degeneration, Hallervorden-Spatz disease, Huntington's disease, ganglioglioma, gangliocytoma, globular glial tauopathy, lead encephalopathy, lipofuscinosis, Lytico-Bodig disease (Parkinson-dementia complex of Guam), meningioangiomatosis, Parkinsonism disease linked to chromosome 17, Pick's disease, primary age-related tauopathy (PART), formerly known as neurofibrillary tangle-only dementia (NFT-dementia), postencephalitic parkinsonism, progressive supranuclear palsy, subacute sclerosing panencephalitis or tuberous sclerosis.

In certain embodiments, the neurodegenerative disease, disorder or condition is selected from Alzheimer's disease, amyotrophic lateral sclerosis, Parkinson's disease and Huntington's disease

In certain embodiments, the injury of the CNS in any one of the above embodiments is selected from spinal cord injury, closed head injury, blunt trauma, penetrating trauma, hemorrhagic stroke, ischemic stroke, cerebral ischemia, optic nerve injury, myocardial infarction, organophosphate poisoning and injury caused by tumor excision.

In certain embodiments, the patient suffering from a neurodegenerative disease, disorder or condition or injury of the CNS is further diagnosed with reduction in cognitive function prior to said treatment, and said indication that the patient is likely to be responsive predicts an improvement in cognitive function.

In certain embodiments, the determining in any one of the above embodiments comprises the steps of: (i) performing an assay on the blood sample of the patient, or fraction thereof, obtained at a time period after a session of treatment with said immune checkpoint modulator to determine one or more of said biomarker selected from (a) to (d); (ii) determining or receiving information of a first reference in a blood sample obtained from the patient, or fraction thereof, before said session of treatment with the immune checkpoint modulator, or from a healthy human population as defined above; (iii) establishing the change for said biomarker by comparing the level of said biomarker with the first reference; and (iv) determining that the patient is likely to be responsive to treatment with said immune checkpoint modulator when the change established in (iii) is an increased level of any one of said biomarker (a) to (c) or a decreased level of said biomarker (d) as compared to the first reference, or that the patient is likely to be non-responsive to treatment with said immune checkpoint modulator when the change established in (iii) is an equal or decreased level of any one of said biomarker (a) to (c) or a decreased level of said biomarker (d) as compared to said first reference.

In certain embodiments, the determining in any one of the above embodiments comprises the steps of: (i) performing an assay on the blood of the patient, or fraction thereof, at a time period before start of treatment with said immune checkpoint modulator to determine one or more of said biomarker selected from (a) to (d); (ii) determining or receiving information of a second reference in a blood sample obtained from a responder patient population before start of treatment with said immune checkpoint modulator as defined above; (iii) establishing the change for said biomarker by comparing the level of said biomarker with the second reference; and (iv) determining that the patient is likely to be responsive to treatment with said immune checkpoint modulator when the change established in (iii) is an equal or increased level of any one of said biomarker (a) to (c) or a decreased level of said biomarker (d) as compared to the second reference, or that the patient is likely to be non-responsive to treatment with said immune checkpoint modulator when the change established in (iii) is an equal or decreased level of any one of said biomarker (a) to (c) or an increased level of said biomarker (d) as compared to said second reference.

In certain embodiments, the assay is a fluorescence-activated cell sorter (FACS) based assay, wherein e.g. the monocyte subpopulation level of (a) to (c) is determined by measuring relative amount of said cells of said subpopulation in a population of peripheral blood mononuclear cell (PBMCs); or the monocyte subpopulation level is determined by measuring fluorescence intensity of said marker on cells of said monocyte subpopulation. FACS methods are well known in the art and can be performed e.g. according to the teachings of Goetz C, Hammerbeck C, Bonnevier J. (2018) Flow Cytometry Basics for the Non-Expert. Springer International Publishing.

In certain embodiments, the biomarker is a soluble peptide, such as a CCL26. In this case, serum or plasma is prepared from the patient's blood sample or from the reference blood sample and the biomarker is detected and/or quantified by using e.g. an enzyme immunoassay, such as enzyme-linked immunosorbent assay (ELISA) and radioimmunoassay (RIA)/Immunoradiometric assay (IRMA) methods, which are well-known in the art (e.g. Thavasu, P W et al. (1992) Measuring cytokine levels in blood. Importance of anticoagulants, processing, and storage conditions. J Immunol Methods 153:115-124; Engvall, E (1972 Nov. 22). “Enzyme-linked immunosorbent assay, Elisa”. The Journal of Immunology. 109 (1): 129-135).

Methods for preparing serum and plasma from blood are readily available to the person of skill in the art (see e.g. Henry, J B (1979) Clinical Diagnosis and Management by Laboratory Methods, Volume 1, W.B Saunders Company, Philadelphia, Pa., p 60).

In certain embodiments, in case the method indicates that the patient is likely to be responsive, said treatment is initiated or continued; and in case the patient is likely to be non-responsive, said treatment is not initiated or discontinued.

In particular embodiments of any one of the above aspects, the immune checkpoint modulator is selected from an agonistic or antagonistic: (i) antibody, such as a humanized antibody; a human antibody; a functional fragment of an antibody; a single-domain antibody, such as a Nanobody; a recombinant antibody; and a single chain variable fragment (ScFv); (ii) antibody mimetic, such as an affibody molecule; an affilin; an affimer; an affitin; an alphabody; an anticalin; an avimer; a DARPin; a fynomer; a Kunitz domain peptide; and a monobody; (iii) aptamer; and (iv) a small molecule; said immune checkpoint modulator modulates activity of an immune checkpoint selected from PD1-PDL1, PD1-PDL2, CD28-CD80, CD28-CD86, CTLA4-CD80, CTLA4-CD86, ICOS-B7RP1, B7H3, B7H4, B7H7, B7-CD28-like molecule, BTLA-HVEM, KIR-MHC class I or II, LAG3-MHC class I or II, CD137-CD137L, OX40-OX40L, CD27-CD70, CD40L-CD40, TIM3-GAL9, V-domain Ig suppressor of T cell activation (VISTA), STimulator of INterferon Genes (STING), T cell immunoglobulin and immunoreceptor tyrosine-based inhibitory motif domain (TIGIT), A2aR-Adenosine, indoleamine-2,3-dioxygenase (IDO)-L-tryptophan, Siglec-3 (CD33), Siglec-5, Siglec-6, Siglec-7, Siglec-8, Siglec-9, Siglec-10, Siglec-11, Siglec-14, and Siglec-16; and a TRAIL receptor; and said disease, disorder or condition is selected from a neurodegenerative disease selected from Alzheimer's disease, a taupathy, amyotrophic lateral sclerosis, Parkinson's disease and Huntington's disease; primary progressive multiple sclerosis; secondary progressive multiple sclerosis; corticobasal degeneration; Rett syndrome; a retinal degeneration disorder selected from age-related macular degeneration and retinitis pigmentosa; anterior ischemic optic neuropathy; glaucoma; uveitis; depression; trauma-associated stress or post-traumatic stress disorder; frontotemporal dementia; Lewy body dementias; mild cognitive impairments; posterior cortical atrophy; primary progressive aphasia; progressive supranuclear palsy; mild cognitive impairment; and aged-related dementia, or said injury of the CNS is selected from spinal cord injury, closed head injury, blunt trauma, penetrating trauma, hemorrhagic stroke, ischemic stroke, cerebral ischemia, optic nerve injury, myocardial infarction, organophosphate poisoning and injury caused by tumor excision.

In particular embodiments, the immune checkpoint modulator is selected from (i) an antibody selected from: (a) anti-PD-L1 antibody; (b) anti-PD-1 antibody; (c) anti-TIM-3 antibody; (d) anti-ICOS antibody; (e) anti-PD-L2 antibody; (f) anti-CTLA-4 antibody; (g) anti-B7RP1 antibody; (h) anti-CD80 antibody; (i) anti-CD86 antibody; (j) anti-B7-H3 antibody; (k) anti-B7-H4 antibody; (1) anti-BTLA antibody; (m) anti-HVEM antibody; (n) anti-CD137 antibody; (o) anti-CD137L antibody; (p) anti-CD-27 antibody; (q) anti-CD70 antibody; (r) anti-CD40 antibody; (s) anti-CD40L antibody; (t) anti-OX40 antibody; (u) anti-OX40L antibody; (v) anti-killer-cell immunoglobulin-like receptor (KIR) antibody; (w) anti-LAG-3 antibody; (x) anti-CD47 antibody; (y) anti-VEGF-A antibody; (z) anti-CD25 antibody; (aa) anti-GITR antibody; (bb) anti-CCR4 antibody; (cc) anti-4-1BB antibody; (dd) an anti-Siglec-3 (CD33) antibody; (ee) an anti-Siglec-5 antibody; (ff) an anti-Siglec-6 antibody; (gg) an anti-Siglec-7 antibody; (hh) an anti-Siglec-8 antibody; (ii) an anti-Siglec-9 antibody; (jj) an anti-Siglec-10 antibody; (kk) an anti-Siglec-11 antibody; (11) an anti-Siglec-14 antibody; (mm) anti-Siglec-16 antibody; (nn) an anti-TRAIL-R1 antibody; (oo) an anti-TRAIL-R2 antibody; and (pp) any combination of (a) to (pp); (ii) any combination of (a) to (pp) in combination with an adjuvant; (iii) a small molecule selected from (a) a p300 inhibitor; (b) Sunitinib; (c) Polyoxometalate-1 (POM-1); (d) α,β-methyleneadenosine 5′-diphosphate (APCP); (e) arsenic trioxide (As2O3); (f) GX15-070 (Obatoclax); (g) a retinoic acid antagonist; (h) an SIRPα (CD47) antagonist; (i) a CCR4 antagonist; (j) an adenosine receptor antagonist; (k) an adenosine A1 receptor antagonist; (1) an adenosine A2a receptor antagonist; (m) an adenosine A2b receptor antagonist; (n) an A3 receptor antagonist; (o) an antagonist of indoleamine-2,3-dioxygenase; and (p) an HIF-1 regulator; an HIF-1 regulator; (iv) any combination of (iii) (a-p) and (i) (a-pp); (v) a protein selected from (a) Neem leaf glycoprotein (NLGP); and (b) sCTLA-4; (vi) a silencing molecule selected from miR-126 antisense and anti-galectin-1 (Gal-1); (vii) OK-432; (viii) a combination of IL-12 and anti-CTLA-4; (ix) an antibiotic agent; and (x) any combination of (i) to (ix); said neurodegenerative disease, disorder or condition is selected from Alzheimer's disease, amyotrophic lateral sclerosis, Parkinson's disease and Huntington's disease; and said patient is further diagnosed with reduction in cognitive function prior to said treatment, and said indication that the patient is likely to be responsive predicts an improvement in cognitive function.

In particular embodiments, the antibody is an antagonistic anti-PD-L1 antibody or an antagonistic anti-PD-1 antibody.

In particular embodiments, the biomarker is the level of a monocyte subpopulation (CD14⁺ cells) expressing CCR2; the antibody used an immune checkpoint modulator is an antagonistic anti-PD-L1 antibody or an antagonistic anti-PD-1 antibody; the blood sample is obtained from the patient at a time period after start of treatment with said antagonistic anti-PD-L1 antibody or antagonistic anti-PD-1 antibody, and an equal or increased level of said biomarker as compared to the level of said subpopulation in a reference blood sample, or a fraction thereof, obtained from the patient before start of treatment with said immune checkpoint modulator or the level of said biomarker in blood, or a fraction thereof, of a healthy human population indicates that the patient is likely to be responsive to treatment with said antagonistic anti-PD-L1 antibody or antagonistic anti-PD-1 antibody; and a decreased level of said biomarker marker as compared to said second reference indicates that the patient is likely to be non-responsive to treatment with said antagonistic anti-PD-L1 antibody or antagonistic anti-PD-1 antibody. Alternatively, the immune checkpoint modulator is an antagonistic anti-Siglec-3 antibody, an antagonistic anti-TIM3 antibody, an antagonistic anti-ICOS antibody, an agonistic anti-ICOS antibody, an antagonistic anti-PD-L2 antibody or an antagonistic anti-CTLA-4 antibody.

In particular embodiments, the biomarker is the level of a monocyte subpopulation (CD14⁺ cells) expressing CCR2^{high}CX3CR1^{low} to a monocyte subpopulation (CD14⁺ cells) expressing CCR2^{low}CX3CR1^{high}; the antibody used an immune checkpoint modulator is an antagonistic anti-PD-L1 antibody or an antagonistic anti-PD-1 antibody; the blood sample is obtained from the patient at a time period before start of treatment with said antagonistic anti-PD-L1 antibody or antagonistic anti-PD-1 antibody, and an equal or increased level of said biomarker as compared to the level of said subpopulation in a reference blood sample, or a fraction thereof, obtained from the patient before start of treatment with said immune checkpoint modulator or the level of said biomarker in blood, or a fraction thereof, of a healthy human population indicates that the patient is likely to be responsive to treatment with said antagonistic anti-PD-L1 antibody or antagonistic anti-PD-1 antibody; and a decreased level of said biomarker marker as compared to said second reference indicates that the patient is likely to be non-responsive to treatment with said antagonistic anti-PD-L1 antibody or antagonistic anti-PD-1 antibody. Alternatively, the immune checkpoint modulator is an antagonistic anti-Siglec-3 antibody, an antagonistic anti-TIM3 antibody, an antagonistic anti-ICOS antibody, an agonistic anti-ICOS antibody, an antagonistic anti-PD-L2 antibody or an antagonistic anti-CTLA-4 antibody.

In particular embodiments, the biomarker is the level of a CCR2 agonist selected from CCL2, CCL7, CCL13, CCL8, CCL11 and CCL16; the antibody used an immune checkpoint modulator is an antagonistic anti-PD-L1 antibody or an antagonistic anti-PD-1 antibody; the blood sample is obtained from the patient at a time period before start of treatment with said antagonistic anti-PD-L1 antibody or antagonistic anti-PD-1 antibody, and an equal or increased level of said biomarker as compared to the level of said subpopulation in in a reference blood sample, or a fraction thereof, obtained from the patient before start of treatment with said immune checkpoint modulator or the level of said biomarker in blood, or a fraction thereof, of a healthy human population indicates that the patient is likely to be responsive to treatment with said antagonistic anti-PD-L1 antibody or antagonistic anti-PD-1 antibody; and a decreased level of said biomarker marker as compared to said second reference indicates that the patient is likely to be non-responsive to treatment with said antagonistic anti-PD-L1 antibody or antagonistic anti-PD-1 antibody. Alternatively, the immune checkpoint modulator is an antagonistic anti-Siglec-3 antibody, an antagonistic anti-TIM3 antibody, an antagonistic anti-ICOS antibody, an agonistic anti-ICOS antibody, an antagonistic anti-PD-L2 antibody or an antagonistic anti-CTLA-4 antibody.

In particular embodiments, the biomarker is the level of a CCR2 antagonist selected from CCL24 and CCL26; the antibody used an immune checkpoint modulator is an antagonistic anti-PD-L1 antibody or an antagonistic anti-PD-1 antibody; the blood sample is obtained from the patient at a time period before start of treatment with said antagonistic anti-PD-L1 antibody or antagonistic anti-PD-1 antibody, and an decreased level of said biomarker of said biomarker as compared to the level of said subpopulation in a reference blood sample, or a fraction thereof, obtained from the patient before start of treatment with said immune checkpoint modulator or the level of said biomarker in blood, or a fraction thereof, of a healthy human population indicates that the patient is likely to be responsive to treatment with said antagonistic anti-PD-L1 antibody or antagonistic anti-PD-1 antibody; and an increased level of said biomarker marker as compared to said second reference indicates that the patient is likely to be non-responsive to treatment with said antagonistic anti-PD-L1 antibody or antagonistic anti-PD-1 antibody. Alternatively, the immune checkpoint modulator is an antagonistic anti-Siglec-3 antibody, an antagonistic anti-TIM3 antibody, an antagonistic anti-ICOS antibody, an agonistic anti-ICOS antibody, an antagonistic anti-PD-L2 antibody or an antagonistic anti-CTLA-4 antibody.

In particular embodiments, the biomarker is the level of a monocyte subpopulation (CD14⁺ cells) expressing CCR2; the antibody used an immune checkpoint modulator is an antagonistic anti-PD-L1 antibody or an antagonistic anti-PD-1 antibody; the blood sample is obtained from the patient at a time period before start of treatment with said antagonistic anti-PD-L1 antibody or antagonistic anti-PD-1 antibody, and an equal or increased level of said biomarker as compared to the level of said subpopulation in blood of a responder patient population before start of treatment with said antagonistic anti-PD-L1 antibody or antagonistic anti-PD-1 antibody indicates that the patient is likely to be responsive to treatment with said antagonistic anti-PD-L1 antibody or antagonistic anti-PD-1 antibody; and a decreased level of said biomarker marker as compared to said second reference indicates that the patient is likely to be non-responsive to treatment with said antagonistic anti-PD-L1 antibody or antagonistic anti-PD-1 antibody. Alternatively, the immune checkpoint modulator is an antagonistic anti-Siglec-3 antibody, an antagonistic anti-TIM3 antibody, an antagonistic anti-ICOS antibody, an agonistic anti-ICOS antibody, an antagonistic anti-PD-L2 antibody or an antagonistic anti-CTLA-4 antibody.

In particular embodiments, the biomarker is the level of a monocyte subpopulation (CD14⁺ cells) expressing CCR2^{high}CX3CR1^{low} to a monocyte subpopulation (CD14⁺ cells) expressing CCR2^{low}CX3CR1^{high}; the antibody used an immune checkpoint modulator is an antagonistic anti-PD-L1 antibody or an antagonistic anti-PD-1 antibody; the blood sample is obtained from the patient at a time period before start of treatment with said antagonistic anti-PD-L1 antibody or antagonistic anti-PD-1 antibody, and an equal or increased level of said biomarker as compared to the level of said subpopulation in blood of a responder patient population before start of treatment with said antagonistic anti-PD-L1 antibody or antagonistic anti-PD-1 antibody indicates that the patient is likely to be responsive to treatment with said antagonistic anti-PD-L1 antibody or antagonistic anti-PD-1 antibody; and a decreased level of said biomarker marker as compared to said second reference indicates that the patient is likely to be non-responsive to treatment with said antagonistic anti-PD-L1 antibody or antagonistic anti-PD-1 antibody. Alternatively, the immune checkpoint modulator is an antagonistic anti-Siglec-3 antibody, an antagonistic anti-TIM3 antibody, an antagonistic anti-ICOS antibody, an agonistic anti-ICOS antibody, an antagonistic anti-PD-L2 antibody or an antagonistic anti-CTLA-4 antibody.

In particular embodiments, the biomarker is the level of a CCR2 agonist selected from CCL2, CCL7, CCL13, CCL8, CCL11 and CCL16; the antibody used an immune checkpoint modulator is an antagonistic anti-PD-L1 antibody or an antagonistic anti-PD-1 antibody; the blood sample is obtained from the patient at a time period before start of treatment with said antagonistic anti-PD-L1 antibody or antagonistic anti-PD-1 antibody, and an equal or increased level of said biomarker as compared to the level of said subpopulation in blood of a responder patient population before start of treatment with said antagonistic anti-PD-L1 antibody or antagonistic anti-PD-1 antibody indicates that the patient is likely to be responsive to treatment with said antagonistic anti-PD-L1 antibody or antagonistic anti-PD-1 antibody; and a decreased level of said biomarker marker as compared to said second reference indicates that the patient is likely to be non-responsive to treatment with said antagonistic anti-PD-L1 antibody or antagonistic anti-PD-1 antibody. Alternatively, the immune checkpoint modulator is an antagonistic anti-Siglec-3 antibody, an antagonistic anti-TIM3 antibody, an antagonistic anti-ICOS antibody, an agonistic anti-ICOS antibody, an antagonistic anti-PD-L2 antibody or an antagonistic anti-CTLA-4 antibody.

In particular embodiments, the biomarker is the level of a CCR2 antagonist selected from CCL24 and CCL26; the antibody used an immune checkpoint modulator is an antagonistic anti-PD-L1 antibody or an antagonistic anti-PD-1 antibody; the blood sample is obtained from the patient at a time period before start of treatment with said antagonistic anti-PD-L1 antibody or antagonistic anti-PD-1 antibody, and an decreased level of said biomarker of said biomarker as compared to the level of said subpopulation in blood of a responder patient population before start of treatment with said antagonistic anti-PD-L1 antibody or antagonistic anti-PD-1 antibody indicates that the patient is likely to be responsive to treatment with said antagonistic anti-PD-L1 antibody or antagonistic anti-PD-1 antibody; and an increased level of said biomarker marker as compared to said second reference indicates that the patient is likely to be non-responsive to treatment with said antagonistic anti-PD-L1 antibody or antagonistic anti-PD-1 antibody. Alternatively, the immune checkpoint modulator is an antagonistic anti-Siglec-3 antibody, an antagonistic anti-TIM3 antibody, an antagonistic anti-ICOS antibody, an agonistic anti-ICOS antibody, an antagonistic anti-PD-L2 antibody or an antagonistic anti-CTLA-4 antibody.

In particular embodiments, in case the patient is likely to be responsive, said treatment is initiated or continued; and in case the patient is likely to be non-responsive, said treatment is not initiated or discontinued.

In still an additional aspect, the present invention provides a kit for predicting whether a patient diagnosed with a disease, disorder, condition or injury of the CNS is likely to be responsive or non-responsive to treatment with an immune checkpoint modulator, or for assessing the efficacy of an immune checkpoint modulator in treating a patient diagnosed with a disease, disorder, condition or injury of the CNS, said kit comprises reagents useful for determining the patients level of a biomarker selected from: (a) the level of a monocyte subpopulation (CD14⁺ cells) expressing CCR2 or CD204 or a combination thereof, or CCR2 and a marker selected from CD204, igf1, lyve1, Stab-1, Siglec1 and Mrc1, or any combination thereof; (b) the ratio of the level of a monocyte subpopulation (CD14⁺ cells) expressing CCR2^{high}CX3CR1^{low} to a monocyte subpopulation (CD14⁺ cells) expressing CCR2^{low}CX3CR1^{high}; (c) the level of a CCR2 agonist selected from CCL2, CCL7, CCL13, CCL8, CCL11 and CCL16; and (d) the level of a CCR2 antagonist selected from CCL24 and CCL26.

In certain embodiments, the kit comprises an antibody, or antigen-binding fragment thereof, that specifically binds to CCR2; and optionally an antibody, or antigen-binding fragment thereof, that specifically binds to a marker selected from CD204, igf1, lyve1, Stab-1, Siglec1 and Mrc1 or any combination thereof.

### Definitions

The term “CNS function” as used herein refers, inter alia, to receiving and processing sensory information, thinking, learning, memorizing, perceiving, producing and understanding language, controlling motor function and auditory and visual responses, maintaining balance and equilibrium, movement coordination, the conduction of sensory information and controlling such autonomic functions as breathing, heart rate, and digestion.

The terms “cognition”, “cognitive function” and “cognitive performance” are used herein interchangeably and are related to any mental process or state that involves but is not limited to learning, memory, creation of imagery, thinking, awareness, reasoning, spatial ability, speech and language skills, language acquisition and capacity for judgment attention. Cognition is formed in multiple areas of the brain such as hippocampus, cortex and other brain structures. However, it is assumed that long term memories are stored at least in part in the cortex and it is known that sensory information is acquired, consolidated and retrieved by a specific cortical structure, the gustatory cortex, which resides within the insular cortex.

In humans, cognitive function may be measured by any know method, for example and without limitation, by the clinical global impression of change scale (CIBIC-plus scale); the Mini Mental State Exam (MMSE); the Neuropsychiatric Inventory (NPI); the Clinical Dementia Rating Scale (CDR); the Cambridge Neuropsychological Test Automated Battery (CANTAB) or the Sandoz Clinical Assessment-Geriatric (SCAG). Cognitive function may also be measured indirectly using imaging techniques such as Positron Emission Tomography (PET), functional magnetic resonance imaging (fMRI), Single Photon Emission Computed Tomography (SPECT), or any other imaging technique that allows one to measure brain function.

Treatment of CNS injury or disease may comprise preventing or inhibiting neuronal degeneration, promotion of neuronal survival, axonal regeneration and/or sprouting, neurogenesis in an injured spinal cord, and/or promotion of functional recovery, as measured for example by the Basso-Beattie-Bresnahan (BBB) score in rats or the Basso Mouse Scale (BMS) in mice, or promotion of recovery of, or decreased rate of loss of cognitive function, as measured in mice e.g. by Radial-arm water maze (RAWM), T-maze, or Y-maze.

The CNS injury according to any one of the above embodiments may be trauma, such as blunt trauma, penetrating trauma, brain coup or contrecoup, trauma sustained during a neurosurgical operation or other procedure, or stroke such as hemorrhagic stroke or ischemic stroke.

The term “responder patient population” as used herein refers to a patient population characterized by individual patients responding favorably, or having favorable response, to a treatment. For example, a population of patients diagnosed with AD, in which the individual patients respond to a treatment with improved cognitive functions is a responder patient population. In contrast, AD patients who do not respond with improved cognitive functions to the same treatment would be defined as a non-responder patient population.

The term “favorable response” as used herein refers to an improvement in one or more symptoms of a disorder, condition or injury of the CNS, as defined herein above, a patient is affected with, and refer to at least a statistically significant improvement of cognitive ability measured as described above.

For example, a favorable response of a patient affected by dementia to treatment may be improvement in cognitive function, such as improvement in learning, plasticity, and/or long term memory; or reduction in a biomarker such as serum amyloid beta peptides or phosphorylated tau peptides in CSF or blood. The terms “improving” and “enhancing” may be used interchangeably.

The term “learning” relates to acquiring or gaining new, or modifying and reinforcing, existing knowledge, behaviors, skills, values, or preferences.

The term “plasticity” relates to synaptic plasticity, brain plasticity or neuroplasticity associated with the ability of the brain to change with learning, and to change the already acquired memory. One measurable parameter reflecting plasticity is memory extinction.

The term “memory” relates to the process in which information is encoded, stored, and retrieved. Memory has three distinguishable categories: sensory memory, short-term memory, and long-term memory.

The term “long term memory” is the ability to keep information for a long or unlimited period of time. Long term memory comprises two major divisions: explicit memory (declarative memory) and implicit memory (non-declarative memory). Long term memory is achieved by memory consolidation which is a category of processes that stabilize a memory trace after its initial acquisition. Consolidation is distinguished into two specific processes, synaptic consolidation, which occurs within the first few hours after learning, and system consolidation, where hippocampus-dependent memories become independent of the hippocampus over a period of weeks to years.

A favorable response of a patient affected by a motor neuron disease, such as amyotrophic lateral sclerosis (ALS) or an injury causing similar symptoms, may be an improvement in any one of the symptoms of these diseases or injury, such as difficulty walking; doing normal daily activities; tripping and falling; muscle weakness, such as weakness in leg, feet, ankles, or hands; slurred speech or trouble swallowing; muscle cramps; and twitching in arms, shoulders and tongue; or any combination thereof.

A favorable response of a patient affected by a neurodegenerative disease of the CNS affecting the motor system, such as parkinsonian syndrome in general and Parkinson's disease in particular, or an injury causing similar symptoms, may be an improvement in any one of the symptoms of these diseases or injury, such as shaking, rigidity, slowness of movement, and difficulty with walking.

The checkpoints that may be targeted according to the present invention are referred to herein as a pair of an immune check point receptor and its native ligand, except when one partner of the pair is unknown, in which case only the known partner is referred to. For example, PD1, which has two known ligands is referred to herein as “PD1-PDL1” or “PD1-PDL2”, while B7H3, the ligand of which has not yet been identified, is referred to simply by “B7H3”.

In some cases, treatment comprises administering an immune checkpoint modulator by a dosage regime comprising at least two sessions (or courses) of therapy, each session of therapy comprising in sequence a treatment session followed by a non-treatment session (where the immune checkpoint modulator is not administered to the patient).

For example, a dosage regime may comprise at least two courses of therapy, each course of therapy comprising in sequence a treatment session where the immune checkpoint modulator is administered once to the individual followed by a non-treatment period of 14 days or longer where the immune checkpoint modulator is not administered to the individual. In particular, the non-treatment period may be 21 or 28 days; two, three, or four weeks; or two to six months, or longer.

Thus, in cases where the blood sample is obtained from the patient at a time period after a session of treatment with said immune checkpoint modulator according to the present invention, the blood sample may be obtained after the first treatment session or after any one of the following treatment sessions as described above.

For example, the blood sample is obtained from the patient at 6, 12, 24 hours or more after any one of the above-mentioned treatment sessions. In certain embodiments, the sample is obtained at 30, 36, 40, 48, 50, 60, 72 hours or more, including up to one week, after any one of the treatment sessions.

In case the blood sample is obtained before treatment started (i.e. before a first treatment session), the blood sample is obtained up to two weeks before start of treatment, e.g. two weeks, or one week, 6, 5, 4, 3, 2, or 1 day or less, up to before the moment of actual administering of the immune checkpoint modulator.

As used herein, the terms “subject” or “individual” or “animal” or “patient” or “mammal,” refers to any subject, particularly a mammalian subject, for whom diagnosis, prognosis, or therapy is desired, for example, a human

The term “treating” as used herein refers to means of obtaining a desired physiological effect. The effect may be therapeutic in terms of partially or completely curing a disease and/or symptoms attributed to the disease. The term refers to inhibiting the disease, i.e. arresting its development; or ameliorating the disease, i.e. causing regression of the disease

The act of obtaining a blood sample from the patient according to the present invention includes directly drawing blood from the patient or receiving the blood sample from a third party that has previously drawn the blood sample from the patient.

The term “fraction of a blood sample” as used herein e.g. in the context of “a blood sample obtained from the patient, or in a fraction thereof, . . . ”, refers to blood plasma or serum as well as sub-populations of cells isolated from the blood, such as PBMCs or monocytes.

The term “peripheral blood mononuclear cell (PBMC)” as used herein refers to any blood cell having a round nucleus, such as a lymphocyte, a monocyte or a macrophage. Methods for isolating PBMCs from blood are readily apparent to those skilled in the art. A non-limiting example is the extraction of these cells from whole blood using ficoll, a hydrophilic polysaccharide that separates layers of blood, with monocytes and lymphocytes forming a buffy coat under a layer of plasma or by leukapheresis, the preparation of leukocyte concentrates with the return of red cells and leukocyte-poor plasma to the donor.

Unless otherwise indicated, all numbers expressing levels of cells, subpopulations of cells, or amount or length of time, are to be understood as being modified in all instances by the term “about”. Accordingly, unless indicated to the contrary, the numerical parameters set forth in this description and attached claims are approximations that may vary by up to plus or minus 10% depending upon the desired properties sought to be obtained by the present invention.

The term “statistically significant difference” as used herein refers to a difference between two groups determined by statistical hypothesis testing as taught for example in Sirkin, R. Mark (2005). “Two-sample t tests”. Statistics for the Social Sciences (3rd ed.). Thousand Oaks, Calif.: SAGE Publications, Inc. pp. 271-316. ISBN 978-1-412-90546-6; or Borror, Connie M. (2009). “Statistical decision making”. The Certified Quality Engineer Handbook (3rd ed.). Milwaukee, Wis.: ASQ Quality Press. pp. 418-472. ISBN 978-0-873-89745-7.

“The terms “a,” “an,” “the” and similar references used in the context of describing the present invention (especially in the context of the following claims) are to be construed to cover both the singular and the plural.” Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of” As such embodiments described herein or so claimed with the phrase “comprising” are expressly or inherently unambiguously described, enabled and supported herein for the phrases “consisting essentially of” and “consisting of.”

The invention will now be illustrated by the following non-limiting Examples.

### EXAMPLES

Material and Methods

Animals. Heterozygous 5×FAD transgenic mice (1g6799; on a C57/BL6-SJL background) co-overexpress mutant forms of human APP associated with familial AD, the Swedish mutation (K670N/M671L), the Florida mutation, (1716V), and the London mutation (V717I). Heterozygous DM-hTAU transgenic mice expressing two mutations K257T/P301S (double mutant, DM; on a BALB-C57/BL6 background) under the natural TAU promoter, associated with severe disease manifestations of frontotemporal-dementia in humans, were kindly provided by Prof. Dan Frenkel Genotyping was performed by polymerase chain reaction (PCR) analysis of tail DNA, as previously described¹⁶. Throughout the study, wild type (WT) controls in each experiment, were non-transgene littermates from the relevant tested mouse colonies. C57BL/6 CD45.2 Ub-GFP mice, in which GFP is ubiquitously expressed²⁴ were used as donors for bone-marrow chimeras. MSR1^{-/-} were generated by Professor Tatsuhiko Kodama, and were kindly provided by Prof. Dan Frenkel¹⁷. Animals were bred and maintained by the Animal Breeding Center of the Weizmann Institute of Science. All experiments detailed herein complied with the regulations formulated by the Institutional Animal Care and Use Committee (IACUC) of the Weizmann Institute of Science.

RNA purification, cDNA synthesis, and quantitative real-time PCR analysis. Total RNA of the hippocampal dentate gyrus (DG) was extracted with TRI Reagent (Molecular Research Center) and purified from the lysates using a RNeasy Kit (Qiagen). The expression of specific mRNAs was assayed using fluorescence-based quantitative real-time PCR (RT-qPCR). RT-qPCR reactions were performed using Fast-SYBR PCR Master Mix (Applied Biosystems). Quantification reactions were performed in triplicate for each sample using the standard curve method. Peptidylprolyl isomerase A (ppia) was chosen as a reference (housekeeping) gene. The amplification cycles were 95° C. for 5 s, 60° C. for 20 s, and 72° C. for 15 s. At the end of the assay, a melting curve was constructed to evaluate the specificity of the reaction. All RT-qPCR reactions were performed and analyzed using StepOne software V2.2.2 (Applied Biosystems).

The following primers were used for the genes indicated:

ppia
forward
[SEQ ID NO: 1]
5′-AGCATACAGGTCCTGGCATCTTGT-3′
and
reverse
[SEQ ID NO: 2]
5′-CAAAGACCACATGCTTGCCATCCA-3′;
tnf-a
forward
[SEQ ID NO: 3]
5′-GCCTCTTCTCATTCCTGCTT-3′
reverse
[SEQ ID NO: 4]
CTCCTCCACTTGGTGGTTTG-3′;
il-12p40
forward
[SEQ ID NO: 5]
5′-GAAGTTCAACATCAAGAGCA-3′
and
reverse
[SEQ ID NO: 6]
5′-CATAGTCCCTTTGGTCCAG-3′;
il-10
forward
[SEQ ID NO: 7]
5′-TGAATTCCCTGGGTGAGAAGCTGA-3′
and
reverse
[SEQ ID NO: 8]
5′-TGGCCTTGTAGACACCTTGGTCTT-3′;
il-6
forward
[SEQ ID NO: 9]
5′-AACAAGAAAGACAAAGCCAG-3′
and
reverse
[SEQ ID NO: 10]
5′-GGAGAGCATTGGAAATTGG-3′;
Il-1β
forward
[SEQ ID NO: 11]
5′-CCAAAAGATGAAGGGCTGCTT-3′
and
reverse
[SEQ ID NO: 12]
5′-TGCTGCTGCGAGATTTGAAG-3′;

Immunohistochemistry. Mice were transcardially perfused with Phosphate Buffered Saline (PBS) before tissue excision and fixation. Tissues that were not adequately perfused wNere not further analyzed, to eliminate autofluorescence associated with blood contamination. Two different tissue preparation protocols (paraffin-embedded or microtome free-floating sections) were applied, as previously described^{9,11}. The following primary antibodies were used: mouse anti-AP (1:300, Covance, #SIG-39320; rabbit anti-GFAP (1:200, Dako, #Z0334); chicken anti-GFAP (1:400, Abcam, #4674); rabbit anti-cleaved caspase 3 (1:100, cell-signaling, #9664); mouse anti-AT-100 and AT-180 (1:50, Innogenetics, #90209 and #90337); mouse anti-Neu-N (1:100, Millipore, #MAB377); rabbit anti-synaptophysin (1:100, Abcam, #32127); mouse anti-Vglutl (1:100, Millipore, MAB5502); goat anti-IBA-1 (1:100, Abcam #5076); rabbit anti-IBA-1 (1:200, Wako #019-19741); mouse anti-IBA-1 (1:100, GeneTex, #GTX632426); rabbit anti-GFP (1:100, MBL, #598); goat anti-GFP (1:100, Abcam, #6658); rabbit anti-IL1f3 (1:100, Santa Cruz Biotechnology, SC-7884); goat anti-IL-10 (1:50, R&D systems, Minneapolis, Minn., #AF519); rabbit anti-MSR1 (1:100, GeneTex, #GTX51749. Secondary antibodies included: Cy2/Cy3/Cy5-conjugated donkey anti-mouse/goat/rabbit/rat antibodies (1:200; all from Jackson Immunoresearch). The slides were exposed to DAPI for nuclear staining (1:10,000; Biolegend) for 1 min. Two negative controls were routinely used in immunostaining procedures, staining with isotype control antibody followed by secondary antibody, or staining with secondary antibody alone. The tissues were applied to slides, mounted with Immu-mount (9990402, from Thermo Scientific), and sealed with cover-slips. Microscopic analysis was performed using a fluorescence microscope (E800; Nikon) or laser-scanning confocal microscope (Zeiss, LSM880). The fluorescence microscope was equipped with a digital camera (DXM 1200F; Nikon), and with either a 20×NA 0.50 or 40×NA 0.75 objective lens (Plan Fluor; Nikon). Recordings were made on postfixed tissues using acquisition software (NIS-Elements, F3 [Nikon] or LSM [Carl Zeiss, Inc.]). For quantification of staining intensity, total cell and background staining were measured using ImageJ software (NIH), and the intensity of specific staining was calculated, as previously described⁵. Images were cropped, merged, and optimized using Photoshop CS6 13.0 (Adobe), and were arranged using Illustrator CS5 15.1 (Adobe).

Radial-arm water maze (RAWM). The RAWM task was used to test spatial learning and memory, as was previously described in detail²⁶. Briefly, six stainless-steel inserts were placed in the tank, forming six swim arms radiating from an open central area. The escape platform was located at the end of one arm (the goal arm), 1.5 cm below the water surface, in a pool 1.1 m in diameter. The water temperature was maintained between 21 and 22° C. Water was made opaque with milk powder. Within the testing room, only distal visual shape and object cues were available to the mice to aid in location of the submerged platform. The goal arm location remained constant for a given mouse. On day 1, mice were trained for 15 trials (spaced over 3 h), with trials alternating between a visible and hidden platform, and the last 4 trails with hidden platform only. On day 2, mice were trained for 15 trials with the hidden platform. Entry into an incorrect arm, or failure to select an arm within 15s, was scored as an error. Spatial learning and memory were measured by counting the number of arm entry errors or the escape latency of the mice on each trial. Training data were analyzed as the mean errors or escape latency, for training blocks of three consecutive trials. The investigator was blind to the identity of the animals throughout the experiments. Data were analyzed and codes were opened by a member of the team who did not perform the behavioral tests.

T-maze. The T-maze test assesses spatial short-term memory and alternation behavior, analyzing the animals' ability to recognize and differentiate between a novel unknown versus a familiar compartment^{16,27}. The T-shaped maze was made of plastic with two 45 cm long arms, which extended at right-angles from a 57 cm long alley. The arms had a width of 10 cm and were surrounded by 10 cm high walls. The test consisted of two trials at an interval of 5 min, during which time the animals were returned to their home cages. During an 8 min acquisition trial, one of the short arms was closed. In the 3 min retention trial, mice had access to both arms and to the alley. Time spent in each of the arms and in the long alley was assessed. Cognitively healthy mice tend to spend more time in the novel arm than in the familiar one or in the alley. Data were recorded using the EthoVision XT 11 automated tracking system (Noldus Information Technology). The investigator was blind to the identity of the animals throughout the experiments. Data were analyzed and codes were opened by a member of the team who did not perform the behavioral tests.

Y-maze. Spontaneous alternation behavior was recorded in a Y-maze to assess short-term memory performance²⁸. The apparatus was a symmetrical Y-maze; each arm measured 50×10 cm, with 40-cm high side walls. Mice were placed in the maze and allowed to freely explore for 5 min. Arms were arbitrarily labeled A, B, and C, and the sequence of arm entries was used to assess alternation behavior. An alternation was defined as consecutive entries into all three arms. The number of maximum alternations was therefore the total number of arm entries minus two, and the percentage of alternations was calculated as (actual alternations/maximum alternations) ×100. For example, for arms referred to as A, B, C, if the mouse performed ABCABCABBAB, the number of arm entries would be 11, and the successive alternations: ABC, BCA, CAB, ABC, BCA, CAB. Therefore, the percentage of alternations would be [6/(11−2)]×100=66.7⁸³. Statistical analysis was performed using analysis of variance (ANOVA) and the Fisher's exact test.

Bone marrow chimerism. Bone marrow (BM) chimeras were prepared as previously described⁵. In brief, chimeras were prepared by subjecting gender-matched recipient mice to lethal irradiation (950 rad), directing the beam to the lower part of the body, and avoiding the head. The mice were then reconstituted with 5×10\hat{ }6 GFP-BM cells. The mice were analyzed 5 weeks after BM transplantation (exhibiting an average of 72% chimerism). CNS-infiltrating GFP⁺ myeloid cells were verified to be CD45^{high}/CD11b^{high}, representing monocyte-derived macrophages and not microglia⁶. In order to study the role of MSR1⁺ monocytes derived-macrophages, gender-matched DM-hTAU and WT littermates were subjected to whole body irradiation (950 rad). The mice were then reconstituted either with 5×10\hat{ }6 MSR^{-/-}-BM cells or with WT BM cells, derived from non-transgene age-matched littermates.

Cresyl Violet staining. Fixed brains were sagittally sectioned, with a section thickness of 6 μm. To estimate neuronal survival, Cresyl violet staining was performed to visualize neurons. Pyramidal neurons were counted in each brain from serial sections located 30 μm apart. All cell counting were performed by a researcher who was blind to the identity of the animals.

Therapeutic Antibodies. For PD-1 blockade, PD-1-specific blocking antibody (anti-PD-1; rat IgG2a isotype; clone RPM1-14; BIOXCELL) and isotype control (anti-trinitrophenol; clone 2A3, BIOXCELL) were administered intraperitoneally (i.p.). For PD-L1 blockade, throughout the entire study PD-L1-blocking antibody directed to mouse PD-L1 was used (anti-PD-L1; rat IgG2b isotype; clone 10F.9G2; BIOXCELL) and isotype control anti-keyhole limpet hemocyanin; clone LTF-2; BIOXCELL) were administered i.p. In one experiment anti-human PD-L1 antibody was used, which was produced as follows: The V-gene sequences of the anti-mouse anti-PD-L1 antibody YW243-55-570 (Ref US20100203056A1) was synthesized and cloned onto coding regions for murine IgG2a/VL-K constant domains. Subsequently, the antibody transiently expressed in HEK 293 cells and purified standard protocols²⁹.

Aβ plaque quantitation. From each brain, 6 μm coronal slices were collected, and five sections per mouse were immunostained, from 4-5 different pre-determined depths throughout the region of interest (dentate gyrus or cerebral cortex). Histogram-based segmentation of positively stained pixels was performed using Image-Pro Plus software (Media Cybernetics, Bethesda, Md., USA). The segmentation algorithm was manually applied to each image, in the dentate gyrus area or in cortical layer V, and the percentage of the area occupied by total Aβ immunostaining was determined. Plaque numbers were quantified from the same 6 μm coronal brain slices, and are presented as average number of plaques per brain region, in the region of interest (ROI), identically marked on all slides from all depths and in all animals examined. Prior to quantification, slices were coded to mask the identity of the experimental groups, and were quantified by an observer blinded to the identity of the groups.

Aggregated tau quantitation. After perfusion, hippocampus and cortex tissues were dissected and homogenized in ice-cold buffer A (349.1 mM sucrose, 0.1 mM CaCl₂), 1 mM MgCl₂) supplemented with protease inhibitor cocktail (Sigma; P8340). Homogenates were diluted in TBS with 1% Triton X-100 (Sigma; T8787) supplemented with protease inhibitor cocktail, and were individually measured by Tau Aggregation Assay using a commercially available kit (CisBio; CB-6FTAUPEG) according to the manufacturer's instructions. This assay is based on the fluorescence resonance energy transfer (FRET) immunoassay. Protein concentrations were measured using BCA protein assay kit (Pierce; 23227) according to the manufacturer's instructions.

IL-1β quantitation. Hippocampal tissue homogenates in buffer A supplemented with protease inhibitor cocktail (as described above) were measured by Mouse IL1 beta assay using a commercially available kit (CisBio; CB-62MIL1BPEG) according to the manufacturer's instructions, and normalized to protein concentration. This assay as above, was based on the fluorescence resonance energy transfer (FRET) immunoassay.

Flow cytometry sample preparation and analysis. Mice were transcardially perfused with PBS, and tissues were treated as previously described 9. Brains were dissociated using the gentleMACS dissociator (Miltenyi Biotec). Spleens were mashed with the plunger of a syringe and treated with ACK (ammonium chloride potassium)-lysing buffer to remove erythrocytes. In all cases, samples were stained according to the manufacturers' protocols. All samples were filtered through a 70-μm nylon mesh, and blocked with anti-Fc CD16/32 (1:100; BD Biosciences). The following fluorochrome-labelled monoclonal antibodies were purchased from BD Pharmingen, BioLegend, R&D Systems or eBiosciences, and used according to the manufacturers' protocols: Brilliant-violet 421-conjugated anti-CD45 or CD-4; PE-conjugated anti-CD3 or anti-CD11b; FITC-conjugated anti-CD44 or anti-CD11b; PerCP-Cy5.5-conjugated anti-CD62L; APC-conjugated anti-Ly6C. Cells were analyzed on an LSRII cytometer (BD Biosciences) using FlowJo software. In each experiment, relevant negative control groups, positive controls and single-stained samples for each tissue were used to identify the populations of interest and to exclude other populations.

Sorting of myeloid cells. Cell populations were sorted with FACSAriaIII (BD Biosciences, San Jose, Calif.). Prior to sorting, all samples were filtered through a 40-μm nylon mesh. For the isolation of monocytes-derived macrophages, samples were gated for CD45^{high} and CD11b^{high} (Brilliant-violet-421, 1:150, 30-F11, Biolegend Inc. San Diego, Calif.; APC CD11b, 1:100, M1/70, eBioscience), while excluding doublets. Isolated cells were single cell sorted into 384-well cell capture plates containing 2 μL of lysis solution and barcoded poly(T) reverse-transcription (RT) primers for single-cell RNA-seq³⁰. Four empty wells were designated in each 384-well plate as a no-cell control during data analysis. Immediately after sorting, each plate was spun down to ensure cell immersion into the lysis solution, snap frozen on dry ice, and stored at −80° C. until processing.

Preparation of massively parallel single-cell RNA-seq library (MARS-seq). Single-cell libraries were prepared as previously described³¹. In brief, mRNA from cells sorted into cell capture plates was barcoded, converted into cDNA, and pooled using an automated pipeline. The pooled sample was then linearly amplified by T7 in vitro transcription, and the resulting RNA was fragmented and converted into a sequencing-ready library by tagging the samples with pooled barcodes and Illumina sequences during ligation, RT, and PCR. Each pool of cells was tested for library quality, and concentration was assessed, as described³¹.

Analysis of single cell RNA-seq data. All MARS-seq libraries were sequenced using an Illumina NextSeq 500 at an average sequencing depth of 50,000 reads per cell. Sequences were demultiplexed, mapped and filtered as previously described⁸⁴, extracting a set of unique molecular identifiers (UMIs) per cell. Cells were than clustered using the MetaCell analysis package³². Briefly, informative genes were used to compute cell-to-cell similarity and to build a K-nn graph (k=50) to group cells into cohesive groups (or “meta-cells”). Finally the package uses bootstrapping to derive strongly separated clusters. The MetaCell package is available at https://bitbucket.org/tanaylab/metacell/src

Preparation of peripheral blood mononuclear cells (PBMCs). The following is based on a GE protocol (http://www.gelifesciences.com/cellprep; Instructions 71-7167-00 AG).
1. Dilute the blood sample in balanced salt buffer (e.g., phosphate-buffered saline) at a 1:1 (volume:volume) ratio. For example, dilute 2 mL of blood in 2 mL of buffer. Gently mix with a Pasteur pipette.
2. Thoroughly mix the Ficoll-Paque medium by repeatedly inverting the stock bottle; then, add the medium to a clean, new centrifuge tube.
3. Layer the diluted blood sample on top of the Ficoll-Paque medium, carefully ensuring that the blood and medium do not mix.
4. Centrifuge the tube at room temperature (i.e. 15-25° C.) for 30 minutes at 400 g with the brake off/soft stop.
5. Remove the tube from the centrifuge, noting the visible layers. The top layer contains plasma, the middle layer is composed of Ficoll medium and granulocytes, and the bottom layer comprises erythrocytes. The PBMCs are located between the top plasma layer and the Ficoll medium.
6. Two techniques can be used to isolate the PBMCs at the plasma/Ficoll interface.

a. Use a clean pipette to carefully remove and discard (or save for later use) the upper plasma layer without disturbing the PBMC-containing plasma/Ficoll interface. Then, transfer the PBMCs to a new, clean tube.

OR

b. Insert a clean pipette through the plasma layer and remove the interface layer containing PMBCs. Avoid extracting plasma or medium, which will contaminate the PBMCs. Gently transfer the PBMC layer to a clean, new tube.
7. Estimate the interface volume, add a 3× volume of balanced salt solution, and gently suspend the PBMCs (e.g., for a 1-mL interface, add 3 mL of PBS).
8. Centrifuge the PBMCs at 200 g for 10 minutes at room temperature and remove the resulting supernatant, which contains any contaminating Ficoll medium or platelets/plasma proteins.
9. Repeat steps 7-8 once more to maximize sample purity.

Statistical analysis. The specific tests used to analyze each set of experiments are indicated in the figure legends. Data were analyzed using a two-tailed Student's t-test to compare between two groups; one-way ANOVA was used to compare several groups, followed by the Fisher's exact test post hoc procedure. Data from behavioral tests were analyzed using two-way repeated-measures ANOVA, and Dunnetts' post hoc procedure was used for multiple comparisons. Sample sizes for behavioral studies were chosen with adequate statistical power based on the literature and past experience, and mice were allocated to experimental groups according to age, gender and genotype. Investigators were blinded to the identity of the groups during experiments and outcome assessment. All inclusion and exclusion criteria were pre-established according to IACUC guidelines. Results are presented as mean±s.e.m. In the graphs, y-axis error bars represent s.e.m. Statistical calculations were performed using GraphPad Prism software (GraphPad Software, San Diego, Calif.).

### Example 1: Targeting PD-1/PD-L1 Pathway in a Mouse Model of Tau Pathology Enhances Recruitment of Monocyte-Derived Macrophages to the Brain Parenchyma

In both 5×FAD and J20 mouse models of AD, disease progression is associated with a reduction of CP expression of leukocyte-trafficking molecules^{9,33}. Treatment with anti-PD-1 antibodies results in 5×FAD mice in enhanced recruitment of monocyte-derived macrophages to the brain⁸. These findings prompted us to test whether the observed beneficial effect of targeting PD-L1 on cognitive function and disease pathology in a tau mouse model was also associated with enhanced trafficking of immune cells to the diseased brain¹². To this end, we first tested whether the administration of antibody directed against PD-L1 induced elevation of effector memory T cells in DM-hTAU mice. Analyzing the spleens of DM-hTAU mice 2 weeks after the anti-PD-L1 antibody administration, revealed increased levels of effector memory T cells (T_{EM}; CD44⁺CD62L^{low}) relative to those in IgG-treated mice (FIG. 1a), as evaluated by flow cytometry analysis. We analyzed by flow cytometry DM-hTAU mice to determine whether the treatment facilitated recruitment of monocyte-derived macrophages (CD45^{high}CD11b^{high}) to the brain parenchyma. We found a significant increase in CD45^{high} CD1b^{high} cells in the brains of DM-hTAU mice treated with anti-PD-L1 antibody relative to those treated with the IgG2b isotype control (FIG. 1b). To confirm the lineage of these cells, which we classified as mainly monocyte-derived macrophages based on their high expression of CD45 and CD11b, we repeated this experiment with bone marrow (BM)-chimeric mice, in which the donor BM cells were taken from mice with GFP-labeled hematopoietic cells²⁴. To create such chimera, recipient DM-hTAU mice were conditioned with lethal-dose irradiation, with the radiation beam targeting the lower part of the body while avoiding the head, prior to BM transplantation⁵. Following establishment of chimerism (See Methods), animals were treated with either anti-PD-L1 or with control IgG2b. Analysis of the brains 2 weeks after the administration of the antibody, by flow cytometry, revealed that among the CD45^{high} CD1b^{high} cells, about 50% of the cells were GFP⁺, which was consistent with the extent of the chimerism, and confirmed their identity as infiltrating monocytes, rather than activated resident microglia (FIG. 1c). No GFP⁺ cells were seen among the CD45^{low}CD11b⁺ cells. Notably, we gated only on myeloid cells, GFP⁺CD45⁺CD11b⁺ cells; BM-derived cells that were GFP⁺CD45⁺CD11b⁻ were not analyzed. Treatment with anti-PD-L1 antibody resulted in an approximately 3-fold increase in the frequency of GFP⁺CD45^{high} CD1b^{high} cells, relative to IgG2b-treated control (FIG. 1c). Notably, this number underestimates the number of homing macrophages, since the chimerism was about 50%. The brains from other mice from the same experiment were excised and processed for immunohistochemistry, which revealed the presence of GFP⁺IBA-1⁺ myeloid cells in the cortex of the anti-PD-L1-treated mice (not shown). We also stained brain sections from the same animals for the anti-inflammatory cytokine, IL-10, and observed its colocalization with infiltrating monocyte-derived macrophages, but not with IBA-1⁺GFP⁻ microglia (not shown).

The overall number of monocyte-derived macrophages that infiltrated the brain was low, and the number of those that were GFP⁺ was even lower. Therefore, we further characterized the infiltrating cells by single-cell RNA-seq. We sorted from both IgG2b-treated and anti-PD-L1 treated groups all the CD45^{high}CD11b^{high}, thereby enriching the monocyte-derived macrophages within the analyzed samples. Clustering analysis of 899 cells (not shown) revealed that the infiltrating monocyte-derived macrophages were heterogeneous, and most likely included several activation states (as seen in clusters 5-10); clusters 1-4 represent activated microglia in several states, and clusters 11-12 indicate neutrophils. Analysis of differential genes in each cluster highlighted a unique signature displayed by clusters 5 and 6, distinct from the resident homeostatic or activated microglia (clusters 1-4); the unique signature was manifested by expression of several molecules that could potentially mediate an important function in disease modification (FIG. 1d, e). One such uniquely expressed molecule is the macrophage scavenger receptor 1 (Msr1) (also known as SRA1, SCARA1, or CD204), an important phagocytic receptor required for engulfment of misfolded and aggregated proteins17,18, and found previously by us to be expressed by M2-like infiltrating monocyte-derived macrophages that are needed for spinal cord repair⁶. Notably, these macrophages expressed additional relevant functional molecules, among which are the insulin-like growth factor-1 (igf1) that was previously reported to enhance neurogenesis in the aged brain¹⁹, lymphatic endothelium-specific hyaluronan receptor (lyve1) and the scavenger receptor stabilin-1 (Stab-1) (FIG. 1e), both of which are markers of anti-inflammatory macrophages, associated with wound healing and lymphogenesis⁵⁴. Additional genes, found here to be uniquely expressed by infiltrating monocyte-derived macrophages, are scavenger receptors such as the sialic acid binding Ig like lectin 1 (Siglec1) and the mannose receptor C-type (Mrc1) (FIG. 1e).

### Example 2. DM-hTAU Chimeras Harboring MSR1−/− Bone Marrow Lose the Ability to Respond to PD-L1 Neutralizing Antibody and Fail to Show Improved Cognitive Ability

In light of the reported role of MSR1 in neurodegenerative diseases, we further focused on this scavenger receptor. Using immunohistochemistry, we confirmed the expression of MSR1 by the GFP⁺ (infiltrating) cells (not shown), in line with our previous findings⁸. Finally, to gain insight into the functional impact of MSR1-expressing macrophages on the repair process, we created bone marrow (BM) chimeric DM-hTAU mice, in which the recipients BM was replaced with donor BM taken from MSR1-deficient mice. As controls we used DM-hTAU chimeric mice in which the recipients BM was replaced with BM taken from non-transgene wild type littermates. Two weeks following the chimerism, the mice were examined for cognitive performance using the T-maze task. We also tested WT chimeric mice that received either wild type BM or BM from MSR1^{-/-} mice (FIG. 1f, g). Following the behavioral test, each group of DM-hTAU chimeric mice was divided into two groups that received either anti-PD-L1 antibody or the control IgG2b, and 4 weeks later were tested again for their performance in the T-maze. Another group of non-chimeric DM-hTAU littermates that received IgG2b control was also evaluated. Anti-PD-L1 reversed cognitive loss in DM-hTAU chimeras harboring BM from wild type mice, while DM-hTAU chimeras harboring MSR1^{-/-} BM lost the ability to respond to PD-L1 neutralizing antibody and failed to show improved cognitive ability (FIG. 1g).

Taken together, our results suggest that systemic immune activation, under conditions of chronic neuroinflammation, associated with murine models of tauopathies, facilitates the entry of monocyte-derived macrophages to the diseased brain and that these cells are key players in the anti-PD-L1 effect on disease modification¹².

### Example 3: Blockade of the PD-1/PD-L1 Axis in a Mouse Model of Alzheimer's Disease Results in Increase of a Specific Monocyte Subpopulation in the Blood

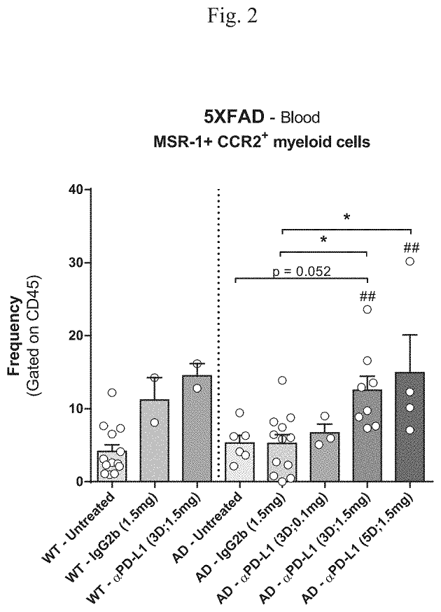

Eight-month old AD or WT mice were treated or not intraperitoneally with either 0.1 mg or 1.5 mg of αPD-L1 or IgG2b and euthanised 3 or 5 days after the administration. Peripheral blood mononuclear cells were isolated and stained for subsequent mass cytometric analysis (CyTOF) (FIG. 2). We found an upregulation of MSR-1+CCR2+ myeloid cell population 3 and 5 days following injection of 1.5 mg αPD-L1, relative to untreated and IgG2b− treated AD groups and relative to untreated WT mice (FIG. 6b).

Animals|Heterozygous 5×FAD transgenic mice (on a C57/BL6-SJL background) that overexpress familial AD mutant forms of human APP (the Swedish mutation, K670N/M671L; the Florida mutation, I716V; and the London mutation, V717I) and PS1 (M146L/L286V) transgenes under the transcriptional control of the neuron-specific mouse Thy-1 promoter5 (5×FAD line Tg6799; The Jackson Laboratory). Genotyping was performed by PCR analysis of tail DNA. Male and female mice were bred and maintained by the animal breeding center of the Weizmann Institute of Science. All experiments detailed herein complied with the regulations formulated by the Institutional Animal Care and Use Committee (IACUC) of the Weizmann Institute of Science.

Mass cytometry (CyTOF)| This method was performed essentially as described in Bendall S C et al, Science. 2011 May 6; 332(6030): 687-696. Briefly, mass cytometry antibodies were either labeled in-house using antibody-labeling kits or purchased from Fluidigm Corporation (South San Francisco, Calif., USA). Antibodies were individually titrated and optimized prior to use. We used cisplatin viability stain prior to proceeding with the cell barcoding of samples with palladium metal isotopes. Briefly, individual samples were fixated and permeabilized and were then incubated with their respective barcodes for 30 minutes at 37° C., after which they were washed with cell staining buffer and combined into composite samples. This was followed by incubation of the composite samples with the cocktail of surface panel antibodies (see chart below) for 30 minutes at 37° C., washing with cell staining and then incubating with intracellular antibodies (see chart below, detailed in bold) for other 30 minutes at 37° C. After washing, samples were incubated with paraformaldehyde 4% overnight at 4° C. Prior to acquisition, samples were washed with cell staining buffer and mass cytometry grade water.

TABLE 1

| Markers | Metal |
|---|---|
| CD45 | Y89 |
| CR5a | 139La |
| Ly6G | 141Pr |
| CD11c | 142Nd |
| GITR | 143Nd |
| CSF1R | 144Nd |
| CD4 | 145Nd |
| F4/80 | 146Nd |
| CD103 | 147Nd |
| CD11b | 148Nd |
| CCR3 | 149Sm |
| CD24 | 150Nd |
| CD25 | 151Eu |
| CD3 | 152Sm |
| CD8 | 153Eu |
| Ter119 | 154Sm |
| NKp46 | 155Gd |
| CD14 | 156Gd |
| Foxp3 | 158Gd |
| PD-1/CD279 | 159Tb |
| CD80 | 160Gd |
| Ki-67 | 161Dy |
| Ly6C | 162Dy |
| CCR6 | 163Dy |
| CX3CR1 | 164Dy |
| PD-L1/CD274 | 165Ho |
| CD63 | 166Er |
| CCR2 | 167Er |
| CR2/CD21 | 168Er |
| Sca1 | 169Tm |
| Siglec-1 | 170Er |
| CD44 | 171Yb |
| MSR-1 | 172Yb |
| CD62L | 173Yb |
| CD209 | 174Lu |
| CD38 | 175Lu |
| B220 | 176Yb |
| MHC-II | 209Bi |

### Example 4: Blockade of CCR2 in Wild Type Mice Leads to Reduction of Myeloid Cell Populations in the Blood without Behavioral Alterations

Treatment with antibodies. For depletion of myeloid cells, the anti-CCR2 antibody, MC21³⁴, was i.p. injected (400 g) every 4 days.

Flow cytometry. Blood was collected from the animals and red blood cells were lysed by ACK Lysing Buffer (Gibco). The samples were washed with PBS, incubated with Fc-block CD16/32 (BD Biosciences), and stained using the following antibodies: FITC-conjugated CD11b, FITC-conjugated CD45, BV421-conjugated CD45, BV421-conjugated CD4, PE-conjugated CD3, APC-conjugated CD44, PerCP-Cy5.5-conjugated CD62L, APC-Cy7-conjugated Ly6G, APC-Cy7-conjugated Ly6G (Biolegend Inc.), PerCP-Cy5.5-conjugated Ly6C and PE-conjugated CD115 (eBioscience, Inc.). The samples were analyzed on a FACS-LSRII cytometer (BD Biosciences) using BD FACSDIVA (BD Biosciences) and FlowJo (FlowJo, LLC) software.

Novel object recognition (NOR). The novel object recognition provides an index of recognition memory⁵. Briefly, mice were placed in a grey, square box (45×45×50 cm) with visual cues on the walls. On habituation day mice were given 20 min to explore the arena without objects. After 24 h, mice were returned for 10 min to the arena in which two similar objects were present in defined locations in the box. Following a break of 60-70 min in home-cage, one of the objects in the arena was exchanged to a novel one, and the mice were returned to the arena for 6 min. Time spent exploring each object was manually scored using EthoVision tracking system XT 11 (Noldus Information Technology), and percentage preference for the novel object was calculated for each animal, by dividing the time spent exploring the novel object by the total exploration time of both objects and multiplying the result by 100%, according to the formula: Percentage preference=((novel object exploration time)/(novel object exploration time+familiar object exploration time))×100%.

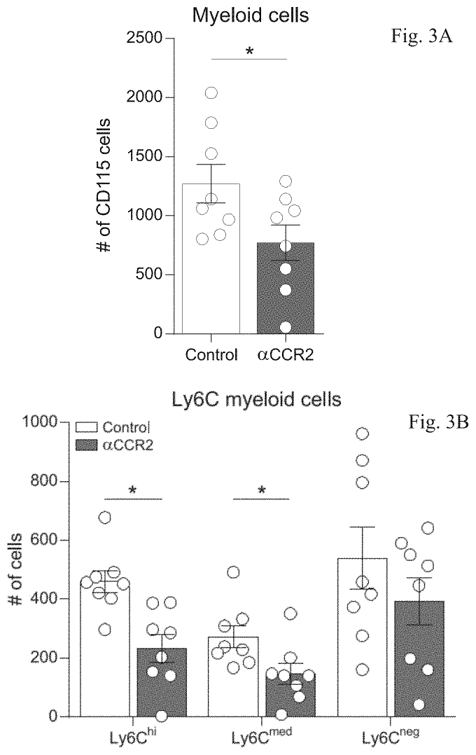

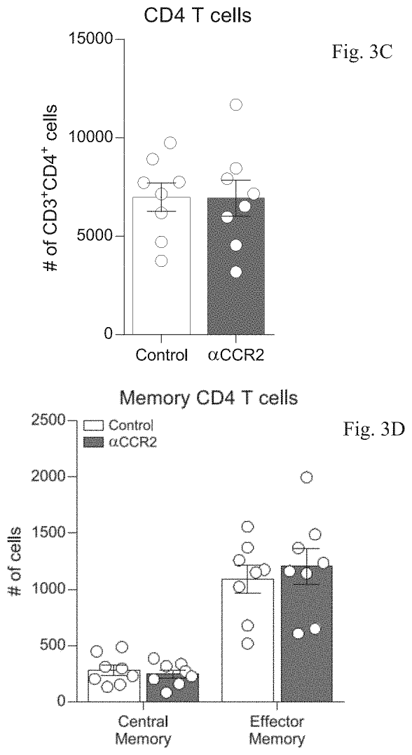

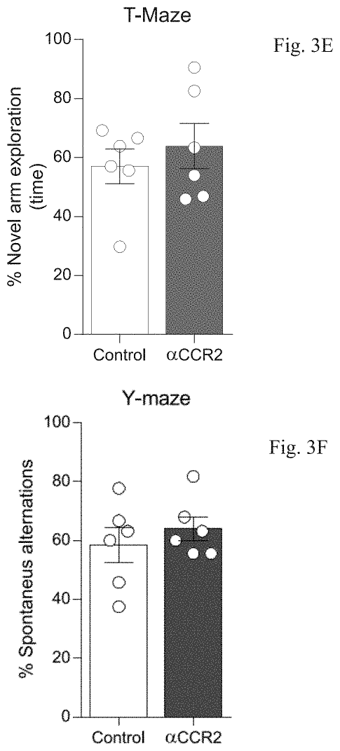

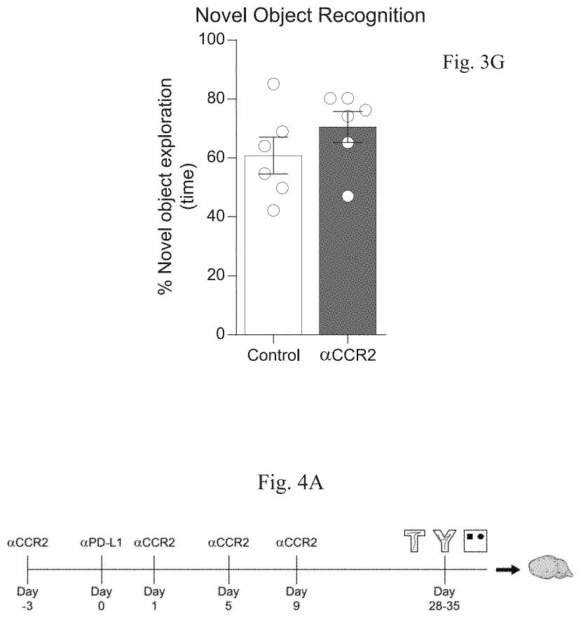

Results. In order to study the involvement of monocytes in the therapeutic effect of the anti-PD-L1 treatment (αPD-L1), we sought for a tool which will allow us blocking or eliminating monocytes. CC chemokine receptor 2 (CCR2) is a chemokine receptor expressed mainly by monocytes, and was shown to play a critical role for monocyte migration from the bone marrow to the blood and for recruitment of inflammatory monocytes into the injured/diseased brain²². MC21 is an anti CCR2 antibody, which was demonstrated to deplete monocytes from the peripheral blood³⁴, thus may be a beneficial tool; however, CCR2 can be expressed by other cell types, including effector memory CD4 T cells³⁵, which play a role in activating the choroid plexus (CP) to express trafficking molecules that allow entry of leukocytes into the brain¹¹. In order to verify the usage of MC21 for our purposes, we analyzed the blood of naïve WT animals following the treatment; every 4 days the animals were intraperitoneally (i.p.) injected with 400 g MC21, to a total of 4 injections and 3 days after the last injection the blood was collected and analyzed by flow cytometry (not shown). Control animals were not treated. We found that the number of CD115⁺Ly6G⁻ myeloid cells was significantly reduced following the treatment, compared to controls (FIG. 3a). Moreover, analysis for Ly6C expressing cells revealed significantly reduced numbers of Ly6C^{med} and Ly6C^{high} monocytes, compared to controls (FIG. 3b). In contrast, analysis for CD4 T cells and for CD4 memory T cell populations did not show any changes following MC21 treatment (FIG. 3c, d). Next, we wished to study whether MC21 treatment has a cognitive effect in WT mice. For this, WT mice were treated with 4-5 injections of MC21, and cognitive assessment for short-term and working memory was performed during the 4 days after the last injection. All three cognitive assessments (novel arm exploration in T-maze, spontaneous alternation in Y-maze and novel object recognition) showed no difference between MC21-treated and control groups (FIG. 3e-g). These results suggest that MC21 treatment reduced monocyte numbers in the blood without modifying neither CD4 T cells populations nor cognitive behavior, and thus, is a suitable tool for our research.

### Example 5: Blockade of CCR2 in a Mouse Model of Tau Pathology Abrogates the Beneficial Effect of PD-L1 Blockade

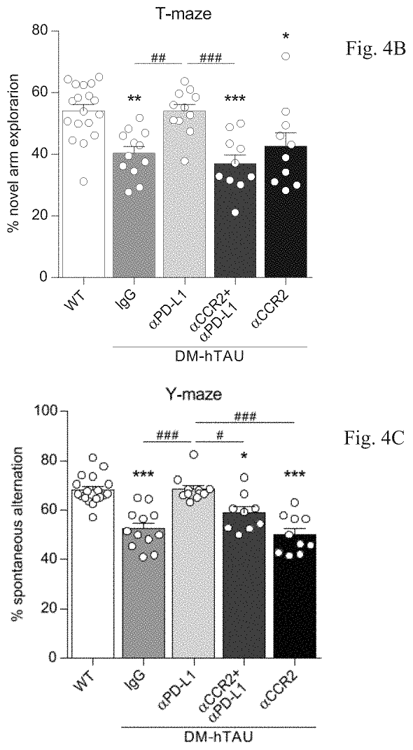

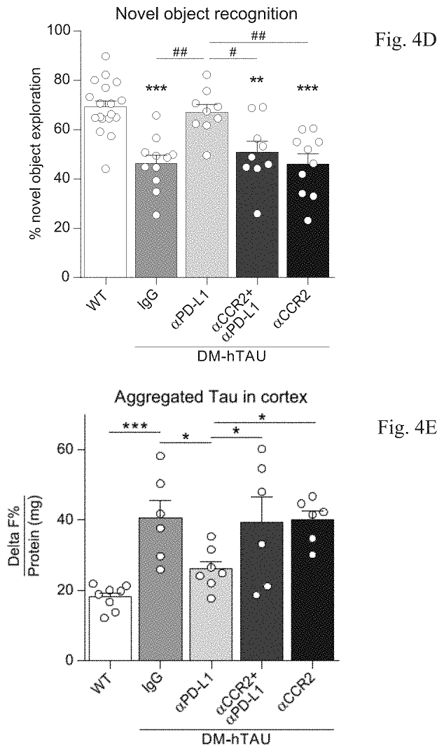

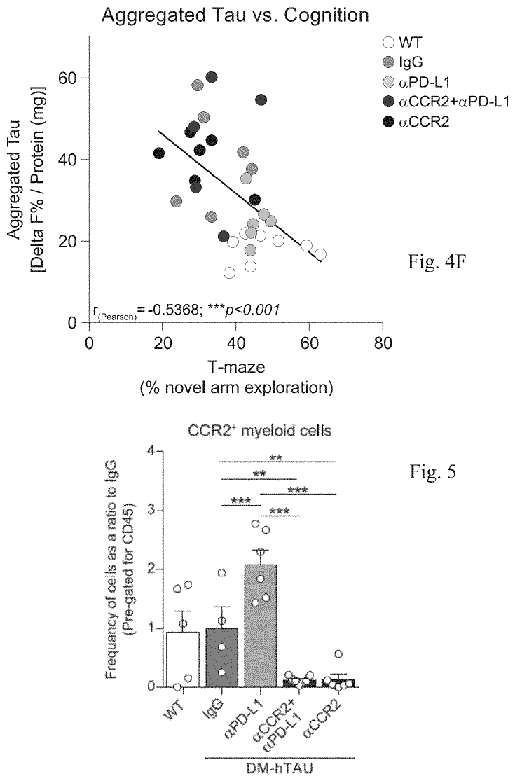

To deplete the monocytes throughout the first 2 weeks of the αPD-L1 treatment, the mice (DM-hTAU of the MC21+PD-L1 group) were injected with MC21 3 days prior the αPD-L1 treatment (day −3), and 3 more times after the treatment (days 1, 5 and 9). The day of αPD-L1 treatment is defined as day 0. Four weeks after the αPD-L1 treatment, cognitive assessment to the animals was performed (novel arm exploration in T-maze, spontaneous alternation in Y-maze and novel object recognition; FIG. 4a). Control IgG group received only anti-IgG antibody injection on day 0, MC21 group received only MC21 injections and the control WT animals were not treated. In all the behavioral paradigms we found that MC21 abrogated the beneficial effect exerted by αPD-L1 treatment in DM-hTAU mice (FIG. 4b-d). Next, we measured aggregated tau protein levels in cortices collected from the mice after the cognitive assessment, using Homogeneous Time Resolved Fluorescence (HTRF) immuno-assay (see Material and Methods). We found in DM-hTAU that αPD-L1 treatment significantly reduced aggregated tau levels in cortices, compared to IgG-treated group and that MC21 treatment abrogated this beneficial effect (FIG. 4e). Moreover, we found a significantly negative correlation between the amount of aggregated tau measured in cortices and the percentage of exploration time of the novel arm in the T-maze (FIG. 4f). Overall, these results suggest that treatment with MC21 abrogated the beneficial effect exerted by αPD-L1.

### Example 6: Blockade of CCR2 in a Mouse Model of Tau Pathology Abolishes the Anti-PD-L1 Antibody Induced Upregulation of CCR2⁺ Myeloid Cells in Blood

Three days following αPD-L1 treatment the blood of the DM-hTAU mice was analyzed by CyTOF (not shown). The cocktail of surface panel antibodies used is shown in Table 2. Quantification of CCR2+ myeloid cells in the blood revealed an upregulation of this population following αPD-L1 treatment. This population was abrogated due to blockade of CCR2 axis (FIG. 5).

TABLE 2

| Marker | Metal | Marker | Metal | Marker | Metal | Marker | Metal |
|---|---|---|---|---|---|---|---|
| CR5a | 139La | Tbet | 160Gd | Ly-6C | 150Nd | Siglec-1 | 170Er |
| Ly6G | 141Pr | CD11c | 161Dy | CD25 | 151Eu | CD44 | 171Yb |
| CD86 | 142Nd | Ki67 | 162Dy | CD3e | 152Sm | MSR-1 | 172Yb |
| IL-4R | 143Nd | CCR6 | 163Dy | PD-L1 | 153Eu | CD62L | 173Yb |
| CD115 | 144Nd | Ly-6A/E | 164Dy | Ter119 | 154Sm | CD209 | 174Yb |
| CD4 | 145Nd | TCRg/d | 165Ho | CD127 (IL-7R) | 155Gd | CD38 | 175Lu |
| cd8a | 146Nd | F4/80 | 166Er | PD-1 | 156Gd | B220 | 176Yb |
| CD103 | 147Sm | CCR2 | 167Er | FoxP3 | 158Gd | CD45 | 89Y |
| CD11b | 148Nd | CD40 | 168Er | GATA3 | 159Tb | MHC-II | 209Bi |
| TNFaR1 | 149Sm | CX3CR1 | 169Tm | | | | |

### Example 7. Blockade of the PD-1/PD-L1 Axis in a Mouse Model of Alzheimer's Disease Results in Increase in the Ratio of the Level of a Monocyte Subpopulation Expressing CCR2^{high}CX3CR1^{low} to a Monocyte Subpopulation Expressing CCR2^{low}CX3CR1^{high} in the Blood

Eight-month old AD or WT mice are treated or not intraperitoneally with either 0.1 mg or 1.5 mg of αPD-L1 or IgG2b and euthanised about 3 or 5 days after the administration. Peripheral blood mononuclear cells are isolated and stained for subsequent mass cytometric analysis (CyTOF). We expect to find an upregulation of CCR2^{high}CX3CR1^{low} myeloid cell population and a no change or downregulation of CCR2^{low}CX3CR1^{high} myeloid cell population about 3 and 5 days following injection of 1.5 mg αPD-L1, relative to untreated and IgG2b− treated AD groups and relative to untreated WT mice.

Animals|Heterozygous 5×FAD transgenic mice (on a C57/BL6-SJL background) that overexpress familial AD mutant forms of human APP (the Swedish mutation, K670N/M671L; the Florida mutation, I716V; and the London mutation, V717I) and PS1 (M146L/L286V) transgenes under the transcriptional control of the neuron-specific mouse Thy-1 promoter5 (5×FAD line Tg6799; The Jackson Laboratory). Genotyping is performed by PCR analysis of tail DNA. Male and female mice are bred and maintained by the animal breeding center of the Weizmann Institute of Science. All experiments detailed herein comply with the regulations formulated by the Institutional Animal Care and Use Committee (IACUC) of the Weizmann Institute of Science.

Mass cytometry (CyTOF)| This method is performed essentially as described in Bendall S C et al, Science. 2011 May 6; 332(6030): 687-696. Briefly, mass cytometry antibodies are either labeled in-house using antibody-labeling kits or purchased from Fluidigm Corporation (South San Francisco, Calif., USA). Antibodies are individually titrated and optimized prior to use. We use cisplatin viability stain prior to proceeding with the cell barcoding of samples with palladium metal isotopes. Briefly, individual samples are fixated and permeabilized and are then incubated with their respective barcodes for 30 minutes at 37° C., after which they are washed with cell staining buffer and combined into composite samples. This is followed by incubation of the composite samples with the cocktail of surface panel antibodies (Table 1) for 30 minutes at 37° C., washing with cell staining and then incubating with intracellular antibodies (see Table 1) for another 30 minutes at 37° C. After washing, samples are incubated with paraformaldehyde 4% overnight at 4° C. Prior to acquisition, samples are washed with cell staining buffer and mass cytometry grade water.

### Example 8. Blockade of CCR2 with an Antagonist in a Mouse Model of Tau Pathology Abrogates the Beneficial Effect of PD-L1 Blockade

To deplete the monocytes throughout the first 2 weeks of the αPD-L1 treatment, the mice (DM-hTAU of the MC21+PD-L1 group) are injected with CCL26 or CCL24 3 days prior the αPD-L1 treatment (day −3), and 3 more times after the treatment (days 1, 5 and 9). The day of αPD-L1 treatment is defined as day 0. Four weeks after the αPD-L1 treatment, cognitive assessment to the animals is performed (novel arm exploration in T-maze, spontaneous alternation in Y-maze and novel object recognition). Control IgG group receive only anti-IgG antibody injection on day 0, CCL26 or CCL24 group receive only CCL26 or CCL24 injections and the control WT animals are not treated. It is expected that CCL26 or CCL24 abrogate the beneficial effect exerted by αPD-L1 treatment in DM-hTAU mice. Alternatively, neutralizing antibodies to CCL26 or CCL24 are injected to the mice. It is expected that the beneficial effect exerted by αPD-L1 treatment in DM-hTAU mice is improved in comparison with control animals.

Aggregated tau protein levels may also be tested in cortices collected from the mice after the cognitive assessment, using Homogeneous Time Resolved Fluorescence (HTRF) immuno-assay (see Material and Methods). It is expected that in DM-hTAU, αPD-L1 treatment significantly reduces aggregated tau levels in cortices, compared to IgG-treated group and that CCL26 or CCL24 treatment abrogate this beneficial effect.

### REFERENCES

1 Rapalino, O. et al. Implantation of stimulated homologous macrophages results in partial recovery of paraplegic rats. Nat Med 4, 814-821 (1998).
2 Moalem, G. et al. Autoimmune T cells protect neurons from secondary degeneration after central nervous system axotomy. Nat Med 5, 49-55, doi:10.1038/4734 (1999).
3 Hauben, E. et al. Posttraumatic therapeutic vaccination with modified myelin self-antigen prevents complete paralysis while avoiding autoimmune disease. J Clin Invest 108, 591-599, doi:10.1172/JCI12837 (2001).
4 Ziv, Y. et al. Immune cells contribute to the maintenance of neurogenesis and spatial learning abilities in adulthood. Nat Neurosci 9, 268-275, doi:nn1629 [pii] 10.1038/nn1629 (2006).
Shechter, R. et al. Infiltrating blood-derived macrophages are vital cells playing an anti-inflammatory role in recovery from spinal cord injury in mice. PLoS Med 6, e1000113, doi:10.1371/journal.pmed.1000113 (2009).
6 Shechter, R. et al. Recruitment of beneficial M2 macrophages to injured spinal cord is orchestrated by remote brain choroid plexus. Immunity 38, 555-569, doi:10.1016/j.immuni.2013.02.012 (2013).
7 Baruch, K. et al. Aging. Aging-induced type I interferon response at the choroid plexus negatively affects brain function. Science 346, 89-93, doi:10.1126/science.1252945 (2014).
8 Baruch, K. et al. PD-1 immune checkpoint blockade reduces pathology and improves memory in mouse models of Alzheimer's disease. Nat Med 22, 135-137, doi:10.1038/nm.4022 (2016).
9 Baruch, K. et al. Breaking immune tolerance by targeting Foxp3(+) regulatory T cells mitigates Alzheimer's disease pathology. Nat Commun 6, 7967, doi:10.1038/ncomms8967 (2015).
Kunis, G., Baruch, K., Miller, O. & Schwartz, M. Immunization with a Myelin-Derived Antigen Activates the Brain's Choroid Plexus for Recruitment of Immunoregulatory Cells to the CNS and Attenuates Disease Progression in a Mouse Model of ALS. J Neurosci 35, 6381-6393, doi:10.1523/JNEUROSCI.3644-14.2015 (2015).
11 Kunis, G. et al. IFN-gamma-dependent activation of the brain's choroid plexus for CNS immune surveillance and repair. Brain 136, 3427-3440, doi:10.1093/brain/awt259 (2013).
12 Rosenzweig, N. et al. PD-1/PD-L1 checkpoint blockade harnesses monocyte-derived macrophages to combat cognitive impairment in a tauopathy mouse model. Nat Commun 10, 465, doi:10.1038/s41467-019-08352-5 (2019).
13 Kipnis, J., Cohen, H., Cardon, M., Ziv, Y. & Schwartz, M. T cell deficiency leads to cognitive dysfunction: implications for therapeutic vaccination for schizophrenia and other psychiatric conditions. Proc Natl Acad Sci USA 101, 8180-8185, doi:10.1073/pnas.0402268101 0402268101 [pii] (2004).
14 Lewitus, G. M. & Schwartz, M. Behavioral immunization: immunity to self-antigens contributes to psychological stress resilience. Mol Psychiatry 14, 532-536, doi:mp2008103 [pii] 10.1038/mp.2008.103 (2009).
15 Oakley, H. et al. Intraneuronal beta-amyloid aggregates, neurodegeneration, and neuron loss in transgenic mice with five familial Alzheimer's disease mutations: potential factors in amyloid plaque formation. J Neurosci 26, 10129-10140, doi:26/40/10129 [pii]10.1523/JNEUROSCI.1202-06.2006 (2006).
16 Rosenmann, H. et al. A novel transgenic mouse expressing double mutant tau driven by its natural promoter exhibits tauopathy characteristics. Exp Neurol 212, 71-84, doi:10.1016/j.expneurol.2008.03.007 (2008).
17 Frenkel, D. et al. Scaral deficiency impairs clearance of soluble amyloid-beta by mononuclear phagocytes and accelerates Alzheimer's-like disease progression. Nat Commun 4, 2030, doi:10.1038/ncomms3030 (2013).
18 El Khoury, J. et al. Scavenger receptor-mediated adhesion of microglia to beta-amyloid fibrils. Nature 382, 716-719, doi:10.1038/382716a0 (1996).
19 Lichtenwalner, R. J. et al. Intracerebroventricular infusion of insulin-like growth factor-I ameliorates the age-related decline in hippocampal neurogenesis. Neuroscience 107, 603-613 (2001).
Schledzewski, K. et al. Lymphatic endothelium-specific hyaluronan receptor LYVE-1 is expressed by stabilin-1+, F4/80+, CD11b+ macrophages in malignant tumours and wound healing tissue in vivo and in bone marrow cultures in vitro: implications for the assessment of lymphangiogenesis. J Pathol 209, 67-77, doi:10.1002/path.1942 (2006).
21 Mildner, A. et al. CCR2+Ly-6Chi monocytes are crucial for the effector phase of autoimmunity in the central nervous system. Brain 132, 2487-2500, doi:10.1093/brain/awp144 (2009).
22 Naert, G. & Rivest, S. A deficiency in CCR2+ monocytes: the hidden side of Alzheimer's disease. J Mol Cell Biol 5, 284-293, doi:10.1093/jmcb/mjt028 (2013).
23 Westin, K. et al. CCL2 is associated with a faster rate of cognitive decline during early stages of Alzheimer's disease. PLoS One 7, e30525, doi:10.1371/journal.pone.0030525 (2012).
24 Paul, F. et al. Transcriptional Heterogeneity and Lineage Commitment in Myeloid Progenitors. Cell 163, 1663-1677, doi:10.1016/j.cell.2015.11.013 (2015).
25 Suffner, J. et al. Dendritic cells support homeostatic expansion of Foxp3+ regulatory T cells in Foxp3.LuciDTR mice. J Immunol 184, 1810-1820, doi:10.4049/jimmunol.0902420 (2010).
26 Alamed, J., Wilcock, D. M., Diamond, D. M., Gordon, M. N. & Morgan, D. Two-day radial-arm water maze learning and memory task; robust resolution of amyloid-related memory deficits in transgenic mice. Nat Protoc 1, 1671-1679, doi:10.1038/nprot.2006.275 (2006).
27 Zueger, M. et al. Olfactory bulbectomy in mice induces alterations in exploratory behavior. Neurosci Lett 374, 142-146, doi:10.1016/j.neulet.2004.10.040 (2005).
28 Hughes, R. N. The value of spontaneous alternation behavior (SAB) as a test of retention in pharmacological investigations of memory. Neurosci Biobehav Rev 28, 497-505, doi:10.1016/j.neubiorev.2004.06.006 (2004).
29 Huang, A. et al. A human programmed death-ligand 1-expressing mouse tumor model for evaluating the therapeutic efficacy of anti-human PD-L1 antibodies. Sci Rep 7, 42687, doi:10.1038/srep42687 (2017).
Jaitin, D. A. et al. Massively parallel single-cell RNA-seq for marker-free decomposition of tissues into cell types. Science 343, 776-779, doi:10.1126/science.1247651 (2014).
31 Keren-Shaul, H. et al. A Unique Microglia Type Associated with Restricting Development of Alzheimer's Disease. Cell 169, 1276-1290 e1217, doi:10.1016/j.cell.2017.05.018 (2017).
32 Baran, Y. MetaCell: analysis of single cell RNA-seq data using k-NN graph partitions. bioRxiv 437665 (2018).
33 Mesquita, S. D. et al. The choroid plexus transcriptome reveals changes in type I and II interferon responses in a mouse model of Alzheimer's disease. Brain Behav Immun 49, 280-292, doi:10.1016/j.bbi.2015.06.008 (2015).
34 Wattananit, S. et al. Monocyte-Derived Macrophages Contribute to Spontaneous Long-Term Functional Recovery after Stroke in Mice. J Neurosci 36, 4182-4195, doi:10.1523/JNEUROSCI.4317-15.2016 (2016).
35 Arkestal, K. et al. CCR2 upregulated on peripheral T cells in osteoarthritis but not in bone marrow. Scand J Immunol 88, e12722, doi:10.1111/sji.12722 (2018).

1. A method for predicting whether a patient diagnosed with a disease, disorder, condition or injury of the Central Nervous System (CNS) is likely to be responsive or non-responsive to treatment with an immune checkpoint modulator, said method comprising determining ex vivo, in a blood sample obtained from the patient a biomarker selected from:
(a) the level of a monocyte subpopulation (CD14⁺ cells) expressing C—C chemokine receptor type 2 (CCR2), macrophage scavenger receptor 1 (CD204) or a combination thereof, or CCR2 and a marker selected from insulin-like growth factor-1 (igf1), lymphatic endothelium-specific hyaluronan receptor (lyve1), scavenger receptor stabilin-1 (Stab-1), sialic acid binding Ig like lectin 1 (Siglec1) and mannose receptor C-type (Mrc1), or any combination thereof;
(b) the ratio of the level of a monocyte subpopulation (CD14⁺ cells) expressing CCR2^{high}CX3CR1^{low} to a monocyte subpopulation (CD14⁺ cells) expressing CCR2^{low}CX3CR1^{high};
(c) the level of a CCR2 agonist selected from CCL2, CCL7, CCL13, CCL8, CCL11 and CCL16; and
(d) the level of a CCR2 antagonist selected from CCL24 and CCL26,
wherein an equal or increased level of said biomarker (a) to (c) or a decreased level of said biomarker (d) in the blood sample, or a fraction thereof, as compared to a first or a second reference indicates that the patient is likely to be responsive to treatment with said immune checkpoint modulator, and an equal or decreased level of any one of said biomarker (a) to (c) or an increased level of said biomarker (d) in the blood sample, or a fraction thereof, as compared to said first or second reference indicates that the patient is likely to be non-responsive to treatment with said immune checkpoint modulator, and
in case the blood sample is obtained from the patient prior to treatment with said immune checkpoint modulator, the level of said biomarker in said blood sample, or fraction thereof, is compared with the first reference, which is the level of said biomarker in blood, or a fraction thereof, of a responder patient population before start of treatment with said immune checkpoint modulator; or
in case the blood sample is obtained from the patient after treatment with said immune checkpoint modulator, the level of said biomarker in said blood sample, or fraction thereof, is compared with the second reference, which is the level of said biomarker in a reference blood sample, or a fraction thereof, obtained from the patient before start of treatment with said immune checkpoint modulator or the level of said biomarker in blood, or a fraction thereof, of a healthy human population.

2. A method of assessing efficacy of an immune checkpoint modulator in treating a patient diagnosed with a disease, disorder, condition or injury of the Central Nervous System (CNS), said method comprising determining ex vivo, in a blood sample obtained from the patient a biomarker selected from:
(a) the level of a monocyte subpopulation (CD14⁺ cells) expressing CCR2, CD204 or a combination thereof; or CCR2 and a marker selected from igf1, lyve1, Stab-1, Siglec1 and Mrc1, or any combination thereof;
(b) the ratio of the level of a monocyte subpopulation (CD14⁺ cells) expressing CCR2^{high}CX3CR1^{low} to a monocyte subpopulation (CD14⁺ cells) expressing CCR2^{low}CX3CR1^{high};
(c) the level of a CCR2 agonist selected from CCL2, CCL7, CCL13, CCL8, CCL11 and CCL16; and
(d) the level of a CCR2 antagonist selected from CCL24 and CCL26,
wherein an equal or increased level of said biomarker (a) to (c) or a decreased level of said biomarker (d) in the blood sample, or a fraction thereof, as compared to a first or a second reference indicates that the immune checkpoint modulator is likely to be efficacious in treating said disease, disorder, condition or injury of the CNS in said patient, and
in case the blood sample is obtained from the patient prior to treatment with said immune checkpoint modulator, the level of said biomarker in said blood sample, or fraction thereof, is compared with the first reference, which is the level of said biomarker in blood, or a fraction thereof, of a responder patient population before start of treatment with said immune checkpoint modulator; or
in case the blood sample is obtained from the patient after treatment with said immune checkpoint modulator, the level of said biomarker in said blood sample, or fraction thereof, is compared with the second reference, which is the level of said biomarker in a reference blood sample, or a fraction thereof, obtained from the patient before start of treatment with said immune checkpoint modulator or the level of said biomarker in blood, or a fraction thereof, of a healthy human population.

3. A method for excluding a patient diagnosed with a disease, disorder, condition or injury of the Central Nervous System (CNS) from treatment with an immune checkpoint modulator, said method comprising determining ex vivo, in a blood sample obtained from the patient a biomarker selected from:
(a) the level of a monocyte subpopulation (CD14⁺ cells) expressing CCR2, CD204 or a combination thereof; or CCR2 and a marker selected from igf1, lyve1, Stab-1, Siglec1 and Mrc1, or any combination thereof;
(b) the ratio of the level of a monocyte subpopulation (CD14⁺ cells) expressing CCR2^{high}CX3CR1^{low} to a monocyte subpopulation (CD14⁺ cells) expressing CCR2^{low}CX3CR1^{high};
(c) the level of a CCR2 agonist selected from CCL2, CCL7, CCL13, CCL8, CCL11 and CCL16; and
(d) the level of a CCR2 antagonist selected from CCL24 and CCL26,
wherein an equal or decreased level of any one of said biomarker (a) to (c) or an increased level of said biomarker (d) in the blood sample, or a fraction thereof, as compared to said first or second reference indicates that the patient is likely to be non-responsive to treatment with said immune checkpoint modulator and is therefore excluded from treatment with said immune checkpoint modulator, and
in case the blood sample is obtained from the patient prior to treatment with said immune checkpoint modulator, the level of said biomarker in said blood sample, or fraction thereof, is compared with the first reference, which is the level of said biomarker in blood, or a fraction thereof, of a responder patient population before start of treatment with said immune checkpoint modulator; or
in case the blood sample is obtained from the patient after treatment with said immune checkpoint modulator, the level of said biomarker in said blood sample, or fraction thereof, is compared with the second reference, which is the level of said biomarker in a reference blood sample, or a fraction thereof, obtained from the patient before start of treatment with said immune checkpoint modulator or the level of said biomarker in blood, or a fraction thereof, of a healthy human population.

4. The method of any one of claims 1 to 3, wherein said immune checkpoint modulator is selected from an agonistic or antagonistic:
(i) antibody, such as a humanized antibody; a human antibody; a functional fragment of an antibody; a single-domain antibody, such as a Nanobody; a recombinant antibody; and a single chain variable fragment (ScFv);
(ii) antibody mimetic, such as an affibody molecule; an affilin; an affimer; an affitin; an alphabody; an anticalin; an avimer; a DARPin; a fynomer; a Kunitz domain peptide; and a monobody;
(iii) aptamer; and
(iv) a small molecule.

5. The method of any one of claims 1 to 4, wherein said immune checkpoint modulator modulates activity of an immune checkpoint selected from PD1-PDL1, PD1-PDL2, CD28-CD80, CD28-CD86, CTLA4-CD80, CTLA4-CD86, ICOS-B7RP1, B7H3, B7H4, B7H7, B7-CD28-like molecule, BTLA-HVEM, KIR-MHC class I or II, LAG3-MHC class I or II, CD137-CD137L, OX40-OX40L, CD27-CD70, CD40L-CD40, TIM3-GAL9, V-domain Ig suppressor of T cell activation (VISTA), STimulator of INterferon Genes (STING), T cell immunoglobulin and immunoreceptor tyrosine-based inhibitory motif domain (TIGIT), A2aR-Adenosine, indoleamine-2,3-dioxygenase (IDO)-L-tryptophan, Siglec-3 (CD33), Siglec-5, Siglec-6, Siglec-7, Siglec-8, Siglec-9, Siglec-10, Siglec-11, Siglec-14, and Siglec-16; and a TRAIL receptor.

6. The method of claim 5, wherein said immune checkpoint modulator is selected from (i) an antibody selected from: (a) anti-PD-L1 antibody; (b) anti-PD-1 antibody; (c) anti-TIM-3 antibody; (d) anti-ICOS antibody; (e) anti-PD-L2 antibody; (f) anti-CTLA-4 antibody; (g) anti-B7RP1 antibody; (h) anti-CD80 antibody; (i) anti-CD86 antibody; (j) anti-B7-H3 antibody; (k) anti-B7-H4 antibody; (1) anti-BTLA antibody; (m) anti-HVEM antibody; (n) anti-CD137 antibody; (o) anti-CD137L antibody; (p) anti-CD-27 antibody; (q) anti-CD70 antibody; (r) anti-CD40 antibody; (s) anti-CD40L antibody; (t) anti-OX40 antibody; (u) anti-OX40L antibody; (v) anti-killer-cell immunoglobulin-like receptor (KIR) antibody; (w) anti-LAG-3 antibody; (x) anti-CD47 antibody; (y) anti-VEGF-A antibody; (z) anti-CD25 antibody; (aa) anti-GITR antibody; (bb) anti-CCR4 antibody; (cc) anti-4-1BB antibody; (dd) an anti-Siglec-3 (CD33) antibody; (ee) an anti-Siglec-5 antibody; (ff) an anti-Siglec-6 antibody; (gg) an anti-Siglec-7 antibody; (hh) an anti-Siglec-8 antibody; (ii) an anti-Siglec-9 antibody; (Oj) an anti-Siglec-10 antibody; (kk) an anti-Siglec-11 antibody; (11) an anti-Siglec-14 antibody; (mm) anti-Siglec-16 antibody; (nn) an anti-TRAIL-R1 antibody; (oo) an anti-TRAIL-R2 antibody; and (pp) any combination of (a) to (pp); (ii) any combination of (a) to (pp) in combination with an adjuvant; (iii) a small molecule selected from (a) a p300 inhibitor; (b) Sunitinib; (c) Polyoxometalate-1 (POM-1); (d) α,β-methyleneadenosine 5′-diphosphate (APCP); (e) arsenic trioxide (As2O3); (f) GX15-070 (Obatoclax); (g) a retinoic acid antagonist; (h) an SIRPα (CD47) antagonist; (i) a CCR4 antagonist; (j) an adenosine receptor antagonist; (k) an adenosine A1 receptor antagonist; (1) an adenosine A2a receptor antagonist; (m) an adenosine A2b receptor antagonist; (n) an A3 receptor antagonist; (o) an antagonist of indoleamine-2,3-dioxygenase; and (p) an HIF-1 regulator; an HIF-1 regulator; (iv) any combination of (iii) (a-p) and (i) (a-pp); (v) a protein selected from (a) Neem leaf glycoprotein (NLGP); and (b) sCTLA-4; (vi) a silencing molecule selected from miR-126 antisense and anti-galectin-1 (Gal-1); (vii) OK-432; (viii) a combination of IL-12 and anti-CTLA-4; (ix) an antibiotic agent; and (x) any combination of (i) to (ix).

7. The method of claim 6, wherein said antibody is an antagonistic anti-PD-L1 antibody.

8. The method of claim 6, wherein said antibody is an antagonistic anti-PD-1 antibody.

9. The method of claim 6, wherein said antibody is an antagonistic anti-Siglec-3 antibody.

10. The method of any one of claims 1 to 9, wherein cells of said monocyte cell subpopulation of (a) to (c) further express a marker selected from CX3CR1, Ki67, IBA-1, and Sca, or any combination thereof.

11. The method of any one of claims 1 to 10, wherein said disease, disorder or condition is selected from a neurodegenerative disease selected from Alzheimer's disease, a taupathy, amyotrophic lateral sclerosis, Parkinson's disease and Huntington's disease; primary progressive multiple sclerosis; secondary progressive multiple sclerosis; corticobasal degeneration; Rett syndrome; a retinal degeneration disorder selected from age-related macular degeneration and retinitis pigmentosa; anterior ischemic optic neuropathy; glaucoma; uveitis; depression; trauma-associated stress or post-traumatic stress disorder; frontotemporal dementia; Lewy body dementias; mild cognitive impairments; posterior cortical atrophy; primary progressive aphasia; progressive supranuclear palsy; mild cognitive impairment; and aged-related dementia.

12. The method of claim 11, wherein said neurodegenerative disease, disorder or condition is selected from Alzheimer's disease, amyotrophic lateral sclerosis, Parkinson's disease and Huntington's disease.

13. The method of any one of claims 1 to 10, wherein said injury of the CNS is selected from spinal cord injury, closed head injury, blunt trauma, penetrating trauma, hemorrhagic stroke, ischemic stroke, cerebral ischemia, optic nerve injury, myocardial infarction, organophosphate poisoning and injury caused by tumor excision.

14. The method of claim 12 or 13, wherein said patient is further diagnosed with reduction in cognitive function prior to said treatment, and said indication that the patient is likely to be responsive predicts an improvement in cognitive function.

15. The method of any one of claims 1 to 14, wherein, in case the patient is likely to be responsive, said treatment is initiated or continued; and in case the patient is likely to be non-responsive, said treatment is not initiated or discontinued.

16. A kit for predicting whether a patient diagnosed with a disease, disorder, condition or injury of the Central Nervous System (CNS) is likely to be responsive or non-responsive to treatment with an immune checkpoint modulator, or assessing the efficacy of an immune checkpoint modulator in treating a patient diagnosed with a disease, disorder, condition or injury of the CNS, said kit comprises reagents useful for determining the patients level of a biomarker selected from:
(a) the level of a monocyte subpopulation (CD14⁺ cells) expressing CCR2 and optionally a marker selected from CD204, igf1, lyve1, Stab-1, Siglec1 and Mrc1, or any combination thereof;
(b) the ratio of the level of a monocyte subpopulation (CD14⁺ cells) expressing CCR2^{high}CX3CR1^{low} to a monocyte subpopulation (CD14⁺ cells) expressing CCR2^{low}CX3CR1^{high};
(c) the level of a CCR2 agonist selected from CCL2, CCL7, CCL13, CCL8, CCL11 and CCL16; and
(d) the level of a CCR2 antagonist selected from CCL24 and CCL26.

17. The kit of claim 16, comprising an antibody, or antigen-binding fragment thereof, that specifically binds to CCR2; and optionally an antibody, or antigen-binding fragment thereof, that specifically binds to a marker selected from CD204, igf1, lyve1, Stab-1, Siglec1 and Mrc1 or any combination thereof.


SEQUENCE LISTING (12 sequences)

SEQ ID NO: 1 - DNA, 24 bp, Artificial Sequence
  synthetic
agcatacaggtcctggcatcttgt

SEQ ID NO: 2 - DNA, 24 bp, Artificial Sequence
  synthetic
caaagaccacatgcttgccatcca

SEQ ID NO: 3 - DNA, 20 bp, Artificial Sequence
  synthetic
gcctcttctcattcctgctt

SEQ ID NO: 4 - DNA, 20 bp, Artificial Sequence
  synthetic
ctcctccacttggtggtttg

SEQ ID NO: 5 - DNA, 20 bp, Artificial Sequence
  synthetic
gaagttcaacatcaagagca

SEQ ID NO: 6 - DNA, 19 bp, Artificial Sequence
  synthetic
catagtccctttggtccag

SEQ ID NO: 7 - DNA, 24 bp, Artificial Sequence
  synthetic
tgaattccctgggtgagaagctga

SEQ ID NO: 8 - DNA, 24 bp, Artificial Sequence
  synthetic
tggccttgtagacaccttggtctt

SEQ ID NO: 9 - DNA, 20 bp, Artificial Sequence
  synthetic
aacaagaaagacaaagccag

SEQ ID NO: 10 - DNA, 19 bp, Artificial Sequence
  synthetic
ggagagcattggaaattgg

SEQ ID NO: 11 - DNA, 21 bp, Artificial Sequence
  synthetic
ccaaaagatgaagggctgctt

SEQ ID NO: 12 - DNA, 20 bp, Artificial Sequence
  synthetic
tgctgctgcgagatttgaag